(19) United States
(12) Patent Application Publication
(10) Pub. No.: US 2023/0404813 A1
(43) Pub. Date: Dec. 21, 2023

(54) EXTERNALLY SUPPORTED DRESSING FOR NEGATIVE-PRESSURE THERAPY

(71) Applicant: KCI Manufacturing Unlimited Company, Westmeath (IE)

(72) Inventors: Jonathan G. REHBEIN, San Antonio, TX (US); Luke PERKINS, San Antonio, TX (US); Shervin JAHANIAN, San Antonio, TX (US); Enrique L. SANDOVAL, San Antonio, TX (US)

(21) Appl. No.: 18/035,637

(22) PCT Filed: Oct. 20, 2021
(86) PCT No.: PCT/IB2021/059675

§ 371 Date: May 5, 2023

Related U.S. Application Data
(60) Provisional application No. 63/112,222, filed on Nov. 11, 2020.

(51) Int. Cl. A61F 13/00; A61F 13/02; A61M 1/00

(52) U.S. Cl. CPC A61F 13/00085; A61F 13/0216; A61M 1/915; A61F 2013/00817; A61F 2013/00834

(57) ABSTRACT

Disclosed embodiments may relate to dressings, covers, and/or drapes configured to provide negative-pressure therapy to a tissue site, such as an incision. In some embodiments, the drape or cover may have an external support layer configured to render the cover or drape self-supporting. In some embodiments, the support layer may comprise a skeleton support structure having a plurality of coupled supports. In some embodiments, the skeleton support structure may be non-planar, for example having abase portion configured to support the cover and a projection portion configured to extend over the manifold of the dressing at a height above the base portion. In some embodiments, the skeleton support structure may be removably coupled to an outer surface of the cover or drape, allowing removal after the dressing has been applied to the tissue site.

### CROSS-REFERENCE TO RELATED APPLICATIONS

This application claims the benefit of priority to U.S. Provisional Application No. 63/112,222, filed on Nov. 11, 2020, which is incorporated herein by reference in its entirety.

### TECHNICAL FIELD

This disclosure relates generally to tissue treatment systems and more particularly, but without limitation, to dressings, systems, and methods relating to negative-pressure therapy.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Reduced-pressure therapy may provide a number of benefits, including migration of epithelial and subcutaneous tissues, improved blood flow, and micro-deformation of tissue at a wound site. Together, these benefits can increase development of granulation tissue and reduce healing times.

While the clinical benefits of negative-pressure therapy are widely known, improvements to therapy systems, components, and processes may benefit healthcare providers and patients.

### BRIEF SUMMARY

New and useful systems, apparatuses, and methods for managing tissue sites in a negative-pressure therapy environment are set forth in the appended claims. The following description provides non-limiting, illustrative example embodiments to enable a person skilled in the art to make and use the claimed subject matter.

Disclosed embodiments may relate to dressings, drapes, and/or dressing assemblies configured to provide negative-pressure therapy to a tissue site, such as an incision. In some embodiments, the drape or cover may have an external support layer. For example, the support layer may comprise or consist essentially of a skeleton support structure adhered to an outer surface of the drape or cover. In some embodiments, the skeleton support structure may comprise a plurality of coupled supports. In some embodiments, the skeleton support structure may be removably adhered to the cover, allowing removal of the skeleton support structure after the dressing, drape, orcover has been placed. In some embodiments, the support layer may be configured to be semi-rigid and/or may be configured to render the cover self-supporting. In some embodiments, the support layer may be configured to provide support to the drape or cover around its perimeter, for example around a flange of the cover extending laterally beyond a manifold of the dressing. In some embodiments, the support layer may be non-planar, for example having a base portion configured to support the flange and a projection portion configured to extend over the manifold of the dressing at a height above the base portion. In some embodiments, the skeleton support structure may comprise a longitudinal support and at least two lateral supports, for example with the lateral supports extending from the longitudinal support in proximity to opposite ends of the longitudinal support. In some embodiments, the projection portion of the skeleton support structure may comprise the longitudinal support. In some embodiments, the base portion may comprise one or more perimeter supports configured to support the perimeter of the flange. In some embodiments, the lateral supports may couple the one or more perimeter supports to the longitudinal support.

In some example embodiments, a dressing assembly may comprise: a dressing having a manifold and a cover disposed over the manifold; and a support layer coupled to or on (e.g. directly coupled to) an outer surface of the cover of the dressing. In some embodiments, the support layer may be removably coupled to the cover, for example to a flange of the cover. In some embodiments, the support layer may comprise a skeleton support structure. In some embodiments, the manifold may comprise a first surface and a second surface; and the cover may be disposed over the second surface of the manifold. In some embodiments, the cover may comprise a flange, extending laterally away from the manifold, in proximity to the first surface of the manifold; and the skeleton support structure may be configured to couple to and to support the flange of the cover. In some embodiments, the skeleton support structure may be configured to render the flange self-supporting. In some embodiments, the skeleton support structure may be non-planar. For example, the skeleton support structure may extend from the outer surface of the flange outward beyond the manifold. In some embodiments, the skeleton support structure may comprise a base portion, configured to support the flange, and a projection portion, configured to extend from the outer surface of the flange over the manifold of the dressing. In some embodiments, the skeleton support structure may comprise a longitudinal support and two lateral supports, extending from the longitudinal support to substantially a perimeter of the flange. In some embodiments, each of the two lateral supports may extend from the longitudinal support in proximity to one of two ends of the longitudinal support. In some embodiments, the two lateral supports may each substantially span a width of the cover and/or may couple to the flange in proximity to the perimeter. In some embodiments, the base portion may comprise one or more perimeter supports configured to span the perimeter of the flange, and the lateral supports may couple the base portion to the longitudinal support. In some embodiments, the projection portion may comprise the longitudinal support. In some embodiments, the base portion of the skeleton support structure may comprise portions of the lateral supports and/or the longitudinal support contacting the flange, and the projection portion of the skeleton support structure may comprise portions of the lateral supports and/or longitudinal support disposed over the manifold and/or above the flange or base portion. In some embodiments, the skeleton support structure may be semi-rigid and/or malleable.

In some example embodiments, a dressing assembly may comprise: a dressing having a manifold and a cover disposed over the manifold, wherein the manifold comprises a tissue-facing surface and an outward-facing surface, the cover is disposed over the outward-facing surface of the manifold, and the cover comprises a flange in proximity to the tissue-facing surface of the manifold; and a skeleton support structure removably coupled to an outer surface of the flange. In some embodiments, the skeleton support structure may extend from the outer surface of the flange over the manifold, and may be configured to render the flange semi-rigid and/or self-supporting. In some embodiments, the skeleton support structure may comprise a base portion, configured to support the flange, and a projection portion, configured to extend over the manifold of the dressing. In some embodiments, the projection portion of the skeleton support structure may be coupled to the base portion and may be configured to form a handle for handling the dressing during placement.

In some example embodiments, a dressing assembly may comprise: a dressing having a manifold and a cover disposed over the manifold; and a support layer coupled to an outer surface of the cover of the dressing; wherein the support layer may be substantially planar (e.g. not extending substantially above the outer surface of the cover). In some embodiments, the support layer may comprise a skeleton support structure. In some embodiments, the manifold may comprise a first surface and a second surface; the cover may be disposed over the second surface of the manifold; the cover may comprise a flange, extending laterally away from the manifold, in proximity to the first surface of the manifold; and the support layer may be configured to couple to and to support the flange of the cover. In some embodiments, the support layer may extend around the manifold (e.g. in a plane parallel to and in proximity to the first surface of the manifold), but not extend over the manifold (e.g. not extend over the second surface of the manifold). In some embodiments, the support layer may not extend substantially above the outer surface of the flange. In some embodiments, the support layer may be removably adhered to the flange, while in other embodiments, the support layer may be permanently attached to the flange. Some embodiments may further comprise an upper film layer or drape, and the support layer may be disposed between the outer surface of the flange and the upper film layer. In some embodiments, the support layer may be configured to render the flange self-supporting.

In some example embodiments, a skeleton support structure may be configured for use with a dressing having a manifold and a cover disposed over the manifold, wherein the cover comprises a flange extending laterally beyond the manifold. For example, the skeleton support structure may be configured to render the flange of the cover self-supporting. In some embodiments, the skeleton support structure may comprise a plurality of supports coupled together and configured to removably couple to an outer surface of the flange of the dressing, for example in proximity to a perimeter of the flange. In some embodiments, the skeleton support structure may be non-planar. In some embodiments, the skeleton support structure may be configured to extend from the outer surface of the flange over the manifold. In some embodiments, the skeleton support structure may further comprise a base portion, configured to support the flange, and a projection portion, configured to extend from the outer surface of the flange over the manifold of the dressing. In some embodiments, the projection portion may be coupled to the base portion and configured to form a handle for handling the dressing during placement. In some embodiments, the base portion may comprise a low-tack adhesive configured to removably couple the base portion to the outer surface of the dressing. In some embodiments, the skeleton support structure may comprise: a longitudinal support configured to extend approximately parallel to the longitudinal centerline of the dressing for at least the length of the manifold; and two lateral supports configured to extend from the longitudinal support to substantially the perimeter of the flange. In some embodiments, the projection portion may comprise the longitudinal support. In some embodiments, the base portion may comprise one or more perimeter supports; and the lateral supports may couple the base portion to the longitudinal support.

In some example embodiments, a method for using a dressing, having a manifold, a cover disposed over the manifold, and a skeleton support structure removably coupled to the cover, may comprise: holding the dressing by the skeleton support structure; applying the dressing to a tissue site by using the skeleton support structure to handle the dressing, wherein the skeleton support structure supports the dressing to prevent the cover from folding over during handling; shaping and adhering the dressing to the tissue site to form a seal for negative-pressure therapy; and/or removing the skeleton support structure from the dressing, while leaving the dressing adhered to the tissue site. In some embodiments, the skeleton support structure may be removed after the dressing has been adhered to the tissue site; and removing the skeleton support structure may not substantially weaken the seal of the dressing to the tissue site.

In some example embodiments, a method of manufacturing a dressing assembly may comprise: providing a dressing having a cover disposed over a manifold; forming a skeleton support structure having a base portion, which is configured to support a perimeter of the cover, and a projection portion, which is configured to extend from the base portion over the manifold; and removably attaching the base portion of the skeleton support structure to an upper surface of the cover. In some embodiments, forming a skeleton support structure may further comprise: providing a longitudinal support and two lateral supports; and coupling each of the lateral supports to opposite ends of the longitudinal support. In some embodiments, the two lateral supports may extend from the longitudinal support to the cover. In some embodiments, the cover may comprise a flange extending laterally beyond the manifold, and the two lateral supports may extend from the longitudinal support to the flange. In some embodiments, forming a skeleton support structure having a base portion may comprise providing one or more perimeter supports; configuring the one or more perimeter supports to provide support around the perimeter of the flange; and coupling the one or more perimeter supports to the longitudinal support with the lateral supports. In some embodiments, the projection portion may comprise the longitudinal support.

Objectives, advantages, and a preferred mode of making and using the claimed subject matter may be understood best by reference to the accompanying drawings in conjunction with the following detailed description of illustrative example embodiments.

### BRIEF DESCRIPTION OF THE DRAWINGS

FIG. 1 is a block diagram of an example embodiment of a therapy system that can provide negative-pressure therapy in accordance with this specification;

FIG. 2 is a graph illustrating example pressure control modes that may be associated with some example embodiments of the therapy system of FIG. 1;

FIG. 3 is a graph illustrating another example pressure control mode suitable for some example embodiments of the therapy system of FIG. 1;

FIG. 4 is an exploded, isometric view of an example embodiment of a dressing that may be associated with an example embodiment of the therapy system of FIG. 1;

FIG. 5 is a top view of another example embodiment of a dressing that may be associated with an example embodiment of the therapy system of FIG. 1;

FIG. 6 is a schematic cross-section view illustrating an exemplary system having an exemplary dressing in place on an exemplary tissue site, illustrating additional details that may be associated with some embodiments;

FIG. 7 is an exploded isometric view of another dressing that may be associated with an embodiment of the system of FIG. 1, illustrating additional details that may be associated with some embodiments; and

FIG. 8 is an exploded isometric view of yet another dressing that may be associated with an embodiment of the system of FIG. 1, illustrating additional details that may be associated with some embodiments;

FIG. 9 is an isometric view of an example embodiment of a dressing assembly that may be associated with some example embodiments of the therapy system of FIG. 1;

FIG. 10 is an isometric view of another example embodiment of a dressing assembly that may be associated with some example embodiments of the therapy system of FIG. 1;

FIG. 11 is a top plan view of yet another example embodiment of a dressing assembly that may be associated with some example embodiments of the therapy system of FIG. 1; and

FIG. 12 is an exploded isometric view of still another example embodiment of a dressing assembly that may be associated with some example embodiments of the therapy system of FIG. 1.

### DESCRIPTION OF EXAMPLE EMBODIMENTS

The following description of example embodiments provides information that enables a person skilled in the art to make and use the subject matter set forth in the appended claims, but may omit certain details already well-known in the art. The following detailed description is, therefore, to be taken as illustrative and non-limiting.

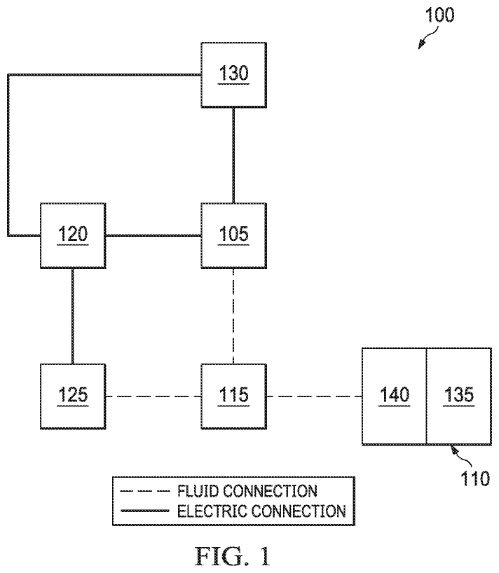

FIG. 1 is a block diagram of an example embodiment of a therapy system 100 that can provide negative-pressure therapy to a tissue site in accordance with this specification. The term “tissue site” in this context may refer to a wound, defect, or other treatment target located on or within tissue, including but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, grafts, and incisions, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

The therapy system 100 may include a source or supply of reduced or negative pressure, such as a negative-pressure source 105, a dressing 110, a fluid container, such as a container 115, and a regulator or controller, such as a controller 120, for example. Additionally, the therapy system 100 may include sensors to measure operating parameters and provide feedback signals to the controller 120 indicative of the operating parameters. As illustrated in FIG. 1, for example, the therapy system 100 may include one or more sensors coupled to the controller 120, such as a first sensor 125 and a second sensor 130. As illustrated in the example of FIG. 1, the dressing 110 may include a tissue interface 135, a cover 140, or both in some embodiments. The dressing 110 may also be referred to as a dressing assembly in some examples, which may include additional or different features as described herein.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 105 may be combined with the controller 120 and other components into a therapy unit.

In general, components of the therapy system 100 may be coupled directly or indirectly. For example, the negative-pressure source 105 may be directly coupled to the container 115, and may be indirectly coupled to the dressing 110 through the container 115. Coupling may include fluid, mechanical, thermal, electrical, or chemical coupling (such as a chemical bond), or some combination of coupling in some contexts. For example, the negative-pressure source 105 may be electrically coupled to the controller 120, and may be fluidly coupled to one or more distribution components to provide a fluid path to a tissue site. In some embodiments, components may also be coupled by virtue of physical proximity, being integral to a single structure, or being formed from the same piece of material.

A distribution component may be detachable, and may be disposable, reusable, or recyclable. The dressing 110 and the container 115 are illustrative of distribution components. A fluid conductor is another illustrative example of a distribution component. A “fluid conductor,” in this context, may include a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include interfaces or fluid ports to facilitate coupling and de-coupling other components. In some embodiments, for example, a dressing interface may facilitate coupling a fluid conductor to the dressing 110. For example, such a dressing interface may be a SENSAT.R.A.C.™ Pad available from KCI of San Antonio, Texas.

A negative-pressure supply, such as the negative-pressure source 105, may be a reservoir of air at a reduced pressure, or may be a manual or electrically-powered device, such as a vacuum pump, a suction pump, a wall suction port available at many healthcare facilities, or a micro-pump, for example. “Negative pressure” or “reduced pressure” generally refers to a pressure less than a local ambient pressure, such as the ambient pressure in a local environment external to a sealed therapeutic environment. In many cases, the local ambient pressure may also be the atmospheric pressure at which a tissue site is located. Further, the pressure may be less than a hydrostatic pressure associated with tissue at the tissue site. Unless otherwise indicated, values of pressure stated herein are gauge pressures. References to increases in reduced pressure may refer to a decrease in absolute pressure, while decreases in reduced pressure may refer to an increase in absolute pressure. While the amount and nature of reduced pressure applied to a tissue site may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The container 115 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 120, may be a microprocessor or computer programmed to operate one or more components of the therapy system 100, such as the negative-pressure source 105. In some embodiments, for example, the controller 120 may be a microcontroller, which may include an integrated circuit containing a processor core and a memory programmed to directly or indirectly control one or more operating parameters of the therapy system 100. Operating parameters may include the power applied to the negative-pressure source 105, the pressure generated by the negative-pressure source 105, or the pressure distributed to the tissue interface 135, for example. The controller 120 may also be configured to receive one or more input signals, such as a feedback signal, and programmed to modify one or more operating parameters based on the input signals.

Sensors, such as the first sensor 125 and the second sensor 130, may be any apparatus operable to detect or measure a physical phenomenon or property, and generally provide a signal indicative of the phenomenon or property that is detected or measured. For example, the first sensor 125 and the second sensor 130 may be configured to measure one or more operating parameters of the therapy system 100. In some embodiments, the first sensor 125 may be a transducer configured to measure pressure in a pneumatic pathway and convert the measurement to a signal indicative of the pressure measured. In some embodiments, for example, the first sensor 125 may be a piezoresistive strain gauge. The second sensor 130 may optionally measure operating parameters of the negative-pressure source 105, such as the voltage or current, in some embodiments. Signals from the first sensor 125 and the second sensor 130 may be suitable as an input signal to the controller 120, but some signal conditioning may be appropriate in some embodiments. For example, the signal may need to be filtered or amplified before it can be processed by the controller 120. Typically, the signal is an electrical signal, but may be represented in other forms, such as an optical signal.

The tissue interface 135 can be adapted to partially or fully contact a tissue site. The tissue interface 135 may take many forms, and may have many sizes, shapes, or thicknesses depending on a variety of factors, such as the type of treatment being implemented or the nature and size of a tissue site. For example, the size and shape of the tissue interface 135 may be adapted to the contours of deep and irregular shaped tissue sites. Moreover, any or all of the surfaces of the tissue interface 135 may have projections or an uneven, course, or jagged profile that can induce strains and stresses on a tissue site, which can promote granulation at the tissue site.

In some embodiments, the tissue interface 135 may be a manifold or may include a manifold and additional layers, components, or features, such as a tissue contact layer, depending on the desired treatment. A “manifold” in this context may include any substance or structure providing a plurality of pathways adapted to collect or distribute fluid relative to a tissue. For example, a manifold may be adapted to receive reduced pressure from a source and distribute reduced pressure through multiple apertures to or from a tissue site, which may have the effect of collecting fluid from a tissue site and drawing the fluid toward the source. In some embodiments, the fluid path may be reversed or a secondary fluid path may be provided to facilitate delivering or moving fluid relative to a tissue site.

In some illustrative embodiments, the pathways of a manifold may be interconnected to improve distribution or collection of fluids at a tissue site. In some illustrative embodiments, a manifold may be a porous foam material having interconnected cells or pores. For example, open-cell foam, porous tissue collections, and other porous material such as gauze or felted mat generally include pores, edges, and/or walls adapted to form interconnected fluid channels. Liquids, gels, and other foams may also include or be cured to include apertures and fluid pathways. In some embodiments, a manifold may additionally or alternatively include projections that form interconnected fluid pathways. For example, a manifold may be molded to provide surface projections that define interconnected fluid pathways.

The average pore size of foam may vary according to needs of a prescribed therapy. For example, in some embodiments, the tissue interface 135 may be foam having pore sizes in a range of 400-600 microns. The tensile strength of the tissue interface 135 may also vary according to needs of a prescribed therapy. For example, the tensile strength of foam may be increased for instillation of topical treatment solutions. In some examples, the tissue interface 135 may be reticulated polyurethane foam such as found in GRANUFOAM™ dressing or V.A.C. VERAFLO™ dressing, both available from KCI of San Antonio, Texas.

The tissue interface 135 may be either hydrophobic or hydrophilic. In an example in which the tissue interface 135 may be hydrophilic, the tissue interface 135 may also wick fluid away from a tissue site, while continuing to distribute negative pressure to the tissue site. The wicking properties of the tissue interface 135 may draw fluid away from a tissue site by capillary flow or other wicking mechanisms. An example of hydrophilic foam is a polyvinyl alcohol, open-cell foam such as V.A.C. WHITEFOAM™ dressing available from KCI of San Antonio, Texas. Other hydrophilic foams may include those made from polyether. Other foams that may exhibit hydrophilic characteristics include hydrophobic foams that have been treated or coated to provide hydrophilicity.

The tissue interface 135 may further promote granulation at a tissue site when pressure within the sealed therapeutic environment is reduced. For example, any or all of the surfaces of the tissue interface 135 may have an uneven, coarse, or jagged profile that can induce microstrain and stress at a tissue site if negative pressure is applied through the tissue interface 135.

In some embodiments, the tissue interface 135 may be constructed from bioresorbable materials. Suitable bioresorbable materials may include, without limitation, a polymeric blend of polylactic acid (PLA) and polyglycolic acid (PGA). The polymeric blend may also include without limitation polycarbonates, polyfumarates, and capralactones. The tissue interface 135 may further serve as a scaffold for new cell-growth, or a scaffold material may be used in conjunction with the tissue interface 135 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

Some embodiments of the tissue interface 135 may comprise layers, components, or features in addition to the manifold. For example, the tissue interface 135 of an absorptive dressing may comprise an absorbent layer, which may be characterized as exhibiting absorbency and/or as being adapted to absorb liquid (such as exudate) from the tissue site. In some embodiments, the absorbent layer may also be adapted to transfer negative pressure therethrough. In some embodiments, the absorbent layer may be configured to retain exudate and/or other fluids drawn from the tissue site during negative-pressure therapy, which may negate the necessity for separate fluid storage components such as an external fluid container. The absorbent layer may comprise any material capable of absorbing liquid (e.g. any absorbent material). In some embodiments, the absorbent layer may exhibit absorbency of at least 3 g saline/g, or at least 5 g saline/g, or from 8 to 20 g saline/g. In some embodiments, the absorbent layer may comprise superabsorbent material, such as superabsorbent polymer (SAP) particles or fibers. For example, some embodiments of the absorbent layer may comprise or consist essentially of one of the following: polyacrylate, sodium polyacrylate, polyacrylamide copolymer, ethylene-maleic anhydride copolymer, polyvinyl alcohol copolymer, cross-linked hydrophilic polymers, and combinations thereof. In some embodiments, the absorbent layer may be hydrophilic. In an example in which the absorbent layer is hydrophilic, the absorbent layer may also absorb or wick fluid away from one or more other components or layers of the dressing 110. In such an embodiment, the wicking properties of the absorbent layer may draw fluid away from one or more components or layers of the dressing 110 by capillary flow or other wicking mechanisms. An example of hydrophilic foam is a polyvinyl alcohol, open-cell foam. Other hydrophilic foams may include those made from polyether. Other foams that may exhibit hydrophilic characteristics include hydrophobic foams that have been treated or coated to provide hydrophilicity.

In some embodiments, the absorbent layer may have a bag-like structure for holding superabsorbent material. For example, the absorbent layer may be configured with superabsorbent material within a wicking pouch. In some embodiments, the pouch may comprise a first wicking layer and a second wicking layer. In some embodiments, the first wicking layer and the second wicking layer may be coupled around the pouch perimeter to form the enclosed pouch encapsulating (e.g. securely holding) the superabsorbent material to contain and prevent the superabsorbent material from migrating out of the pouch. For example, the first and second wicking layers may be coupled to each other using adhesive. The wicking layers may each comprise wicking material. The wicking material may be configured to be permeable to liquid (such as exudate), while retaining the superabsorbent material within the pouch. For example, the porosity of the wicking layers may be sufficiently small to prevent migration of the superabsorbent material through the wicking layers. The wicking layers may be configured to wick liquid along the superabsorbent material in a lateral direction normal to a thickness of the superabsorbent material within the pouch. Wicking of liquid laterally may enhance the distribution of liquid to the superabsorbent material, which may in turn speed absorption and/or allow for the superabsorbent material to maximize its absorbency. Examples of the wicking material may comprise or consist essentially of one of the following: Viscose, PET, Lidro™ non-woven material, a knitted polyester woven textile material, such as the one sold under the name InterDry® AG material from Coloplast A/S of Denmark, GORTEX® material, DuPont Softesse® material, etc., and combinations thereof. In some embodiments, the absorbent layer may serve as the manifold. For example, the absorbent layer may have manifolding properties, such that a separate manifold may not be necessary for negative-pressure therapy.

Some embodiments of the tissue interface 135 may comprise a protective layer (e.g. a tissue-contact layer). In some embodiments, the protective layer may act as a comfort layer, configured to improve comfort at the tissue site. In some embodiments, the protective layer may act as a fluid control layer, configured to minimize maceration, backflow of exudate out of the dressing to the tissue site, and/or tissue in-growth from the tissue site into the dressing 110. The protective layer may be configured to allow fluid transport from the tissue site into the dressing 110 and/or to manifold during negative-pressure therapy. In some embodiments, the protective layer may be configured as the tissue-contact surface for the dressing, so that in use it may be located adjacent to and/or direct contact with the tissue site. In some embodiments, the protective layer may be located between the tissue-contact surface and the manifold and/or the absorbent layer. In some embodiments, the protective layer may be located between the tissue site (when the dressing is in use) and the manifold and/or absorbent layer.

In some embodiments, the protective layer may comprise a porous fabric, a porous film, or a polymeric film (e.g. which may be liquid impermeable) with a plurality of fluid passages (e.g. slits, slots, or fluid valves). In some embodiments, the protective layer may comprise or consist essentially of a woven, elastic material or a polyester knit textile substrate. As a non-limiting example, an InterDry™ textile material from Milliken Chemical of Spartanburg, South Carolina, may be used. The protective layer may also include anti-microbial substances, such as silver, in some embodiments.

In some embodiments, the protective layer may comprise or consist essentially of a liquid-impermeable, elastomeric material. For example, the protective layer may comprise or consist essentially of a polymer film. In some embodiments, for example, the protective layer may comprise or consist essentially of a hydrophobic polymer, such as a polyethylene film. The simple and inert structure of polyethylene can provide a surface that interacts little, if any, with biological tissues and fluids, providing a surface that may encourage the free flow of liquids and low adherence, which can be particularly advantageous for many applications. Other suitable polymeric films include polyurethanes, acrylics, polyolefin (such as cyclic olefin copolymers), polyacetates, polyamides, polyesters, copolyesters, PEBAX block copolymers, thermoplastic elastomers, thermoplastic vulcanizates, polyethers, polyvinyl alcohols, polypropylene, polymethylpentene, polycarbonate, styreneics, silicones, fluoropolymers, and acetates. A thickness between 20 microns and 100 microns may be suitable for many applications. In some embodiments, the protective layer may be hydrophobic. In some embodiments, the protective layer may be hydrophilic. In some embodiments, the protective layer may be suitable for coupling, such as welding, to other layers, such as the manifold.

Some embodiments of the protective layer may have one or more fluid passages, which can be distributed uniformly or randomly across the protective layer. The fluid passages may be bi-directional and pressure-responsive. For example, each of the fluid passages generally may comprise or consist essentially of an elastic passage that is normally unstrained to substantially reduce liquid flow, and can expand or open in response to a pressure gradient. In some embodiments, the fluid passage may comprise or consist essentially of perforations in the protective layer. Perforations may be formed by removing material from the protective layer. For example, perforations may be formed by cutting through the protective layer, which may also deform the edges of the perforations in some embodiments. In the absence of a pressure gradient across the perforations, the passages may be sufficiently small to form a seal or fluid restriction, which can substantially reduce or prevent liquid flow. Additionally or alternatively, one or more of the fluid passages may be an elastomeric valve that is normally closed when unstrained to substantially prevent liquid flow, and can open in response to a pressure gradient. A fenestration may be a suitable valve for some applications. Fenestrations may also be formed by removing material from the protective layer, but the amount of material removed and the resulting dimensions of the fenestrations may be up to an order of magnitude less than perforations, and may not deform the edges.

For example, some embodiments of the fluid passages may comprise or consist essentially of one or more slits, slots or combinations of slits and slots in the protective layer. In some examples, the fluid passages may comprise or consist of linear slots having a length less than 4 millimeters and a width less than 1 millimeter. The length may be at least 2 millimeters, and the width may be at least 0.4 millimeters in some embodiments. A length of about 3 millimeters and a width of about 0.8 millimeters may be particularly suitable for many applications, and a tolerance of about 0.1 millimeter may also be acceptable. Such dimensions and tolerances may be achieved with a laser cutter, for example. Slots of such configurations may function as imperfect valves that substantially reduce liquid flow in a normally closed or resting state. For example, such slots may form a flow restriction without being completely closed or sealed. The slots can expand or open wider in response to a pressure gradient to allow increased liquid flow.

In some embodiments, the cover 140 may provide a bacterial barrier and protection from physical trauma. The cover 140 may also be constructed from a material that can reduce evaporative losses and provide a fluid seal between two components or two environments, such as between a therapeutic environment and a local external environment. For example, the cover 140 may comprise or consist essentially of an elastomeric film or membrane that can provide a seal adequate to maintain a reduced pressure at a tissue site for a given negative-pressure source. In some example embodiments, the cover 140 may be a polymer drape, such as a polyurethane film, that is permeable to water vapor but impermeable to liquid. The cover 140 may have a high moisture-vapor transmission rate (MVTR) in some applications. For example, the MVTR may be at least 250 g/m\hat{ }2 per twenty-four hours in some embodiments (based on ASTM E96/E96M for upright cup measurement, e.g. at 38 degrees Celsius and 10% relative humidity). In some embodiments, an MVTR up to 2600 grams per square meter per twenty-four hours or up to 5,000 grams per square meter per twenty-four hours may provide effective breathability and mechanical properties. Such drapes typically have a thickness in the range of 25-50 microns. For permeable materials, the permeability generally should be low enough that a desired negative pressure may be maintained. In some embodiments, the cover 140 may form an outer surface of the dressing 110.

The cover 140 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; an INSPIRE 2301 and INSPIRE 2327 material from Coveris Advanced Coatings of Wrexham, United Kingdom having, for example, an MVTR (inverted cup technique) of 14400 g/m2/24 hours and a thickness of about 30 microns; a thin, uncoated polymer drape; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); EVA film; co-polyester; a silicone drape; a 3M Tegaderm® drape; a polyurethane drape such as one available from Avery Dennison Corporation of Glendale, California; polyether block polyamide copolymer (PEBAX), for example, from Arkema, France; INSPIRE 2327; or other appropriate material.

An attachment device may be used to attach the cover 140 to an attachment surface, such as undamaged epidermis, a gasket, or another cover (e.g. at the tissue site). The attachment device may take many forms. For example, an attachment device may be a medically-acceptable, pressure-sensitive adhesive configured to bond the cover 140 to epidermis around a tissue site. In some embodiments, for example, some or all of the cover 140 may be coated with an adhesive, such as an acrylic adhesive, which may have a coating weight between 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

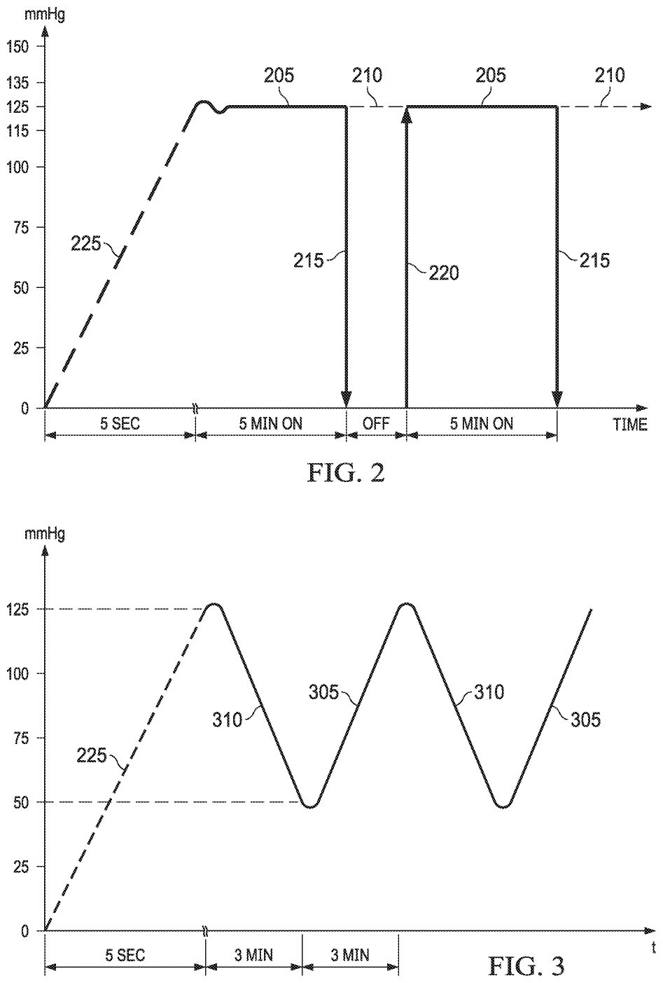

FIG. 2 is a graph illustrating additional details of an example control mode that may be associated with some embodiments of the controller 120. In some embodiments, the controller 120 may have a continuous pressure mode, in which the negative-pressure source 105 is operated to provide a constant target reduced pressure, as indicated by line 205 and line 210, for the duration of treatment or until manually deactivated. Additionally or alternatively, the controller may have an intermittent pressure mode, as illustrated in the example of FIG. 2. In FIG. 2, the x-axis represents time, and the y-axis represents reduced pressure generated by the negative-pressure source 105 over time. In the example of FIG. 2, the controller 120 can operate the negative-pressure source 105 to cycle between a target pressure and atmospheric pressure. For example, the target pressure may be set at a value of 125 mmHg, as indicated by line 205, for a specified period of time (e.g., 5 min), followed by a specified period of time (e.g., 2 min) of deactivation, as indicated by the gap between the solid lines 215 and 220. The cycle can be repeated by activating the negative-pressure source 105, as indicated by line 220, which can form a square wave pattern between the target pressure and atmospheric pressure.

In some example embodiments, the increase in negative-pressure from ambient pressure to the target pressure may not be instantaneous. For example, the negative-pressure source 105 and the dressing 110 may have an initial rise time, as indicated by the dashed line 225. The initial rise time may vary depending on the type of dressing and therapy equipment being used. For example, the initial rise time for one therapy system may be in a range of about 20-30 mmHg/second and in a range of about 5-10 mmHg/second for another therapy system. If the therapy system 100 is operating in an intermittent mode, the repeating rise time as indicated by the solid line 220 may be a value substantially equal to the initial rise time as indicated by the dashed line 225.

FIG. 3 is a graph illustrating additional details that may be associated with another example pressure control mode in some embodiments of the therapy system 100. In FIG. 3, the x-axis represents time and the y-axis represents negative pressure generated by the negative-pressure source 105. The target pressure in the example of FIG. 3 can vary with time in a dynamic pressure mode. For example, the target pressure may vary in the form of a triangular waveform, varying between a minimum and maximum reduced pressure of 50-125 mmHg with a rise time 305 set at a rate of +25 mmHg/min. and a descent time 310 set at −25 mmHg/min, respectively. In other embodiments of the therapy system 100, the triangular waveform may vary between reduced pressure of 25-125 mmHg with a rise time 305 set at a rate of +30 mmHg/min and a descent time 310 set at −30 mmHg/min.

In some embodiments, the controller 120 may control or determine a variable target pressure in a dynamic pressure mode, and the variable target pressure may vary between a maximum and minimum pressure value that may be set as an input prescribed by an operator as the range of desired reduced pressure. The variable target pressure may also be processed and controlled by the controller 120, which can vary the target pressure according to a predetermined waveform, such as a triangular waveform, a sine waveform, or a saw-tooth waveform. In some embodiments, the waveform may be set by an operator as the predetermined or time-varying reduced pressure desired for therapy.

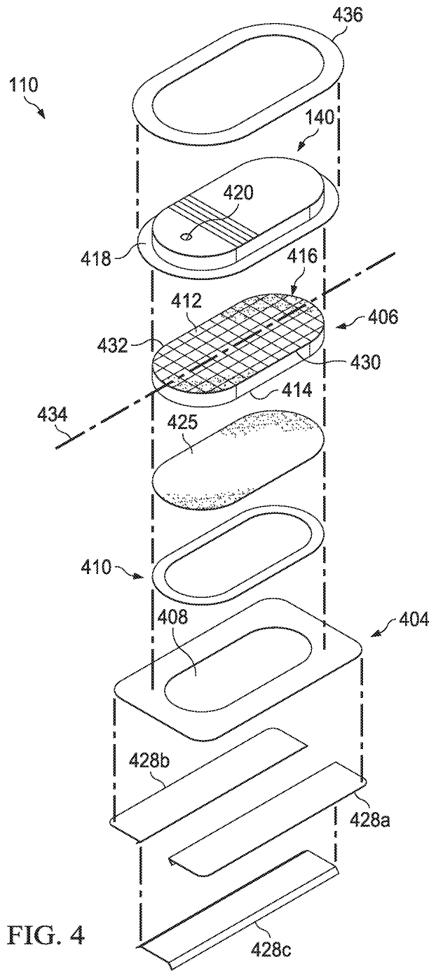

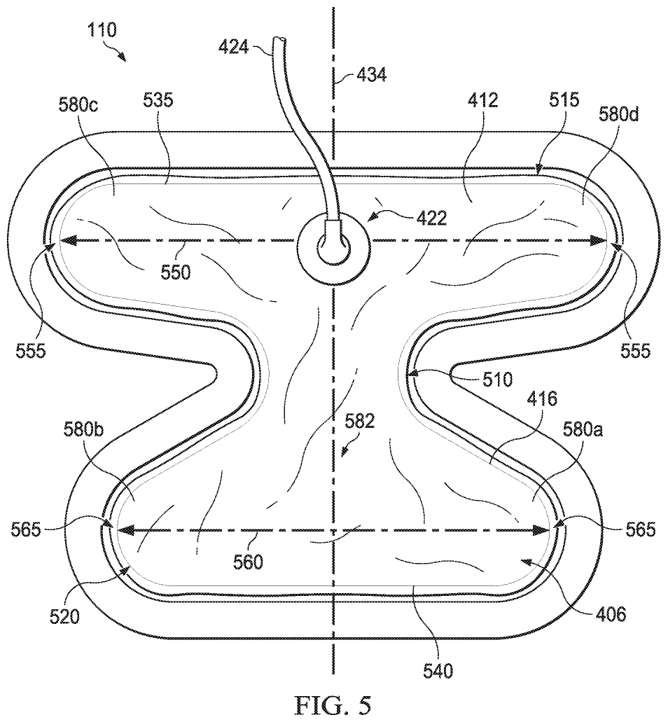

Referring to FIGS. 4-5, the dressing 110 may include features that can treat a tissue site, or parts thereof, and an area of tissue around the tissue/treatment site. For example, the tissue site may be an incision or other treatment target on a patient. The dressing 110 may be configured to treat not only the incision or treatment target, but also, an area of tissue around the incision or treatment target. While the figures may illustrate exemplary dressing embodiments with a particular shape, other exemplary dressings may have other sizes, shapes, and/or configurations, for example for use on other tissue sites.

FIG. 4 is an exploded, isometric view of an example embodiment of a dressing or dressing assemblage that may be associated with an example embodiment of the therapy system of FIG. 1. Referring more specifically to FIG. 4, in some examples, the dressing 110 may include an attachment device 404, a manifold 406, and the cover 140. Some examples of the attachment device 404 and other components may include a treatment aperture 408, and the manifold 406 may be configured to be at least partially exposed to a tissue site through the treatment aperture 408. Further, in some examples, the dressing 110 may optionally include an adhesive ring 410 that may be configured to bond a peripheral portion of the manifold 406 to a portion of the attachment device 404. In some examples, the adhesive ring 410 may be formed as part of the attachment device 404, or the adhesive ring 410 may be omitted with the attachment device 404 instead being coupled to the manifold 406 and/or cover 140 with another medically acceptable coupling apparatus. In some examples, the cover 140, the manifold 406, the optional adhesive ring 410, and the attachment device 404 may have similar shapes. The attachment device 404 may be slightly larger than the manifold 406 to permit coupling of the attachment device 404 to the cover 140 around the manifold 406. In some examples, an adhesive may be disposed on a portion of the manifold 406 exposed through the treatment aperture 408. In some embodiments, the adhesive may be pattern-coated, and may cover up to 50% of the exposed portion or surface of the manifold 406.

The cover 140, the manifold 406, the attachment device 404, or various combinations may be assembled, for example forming a pre-assembled dressing assemblage, before application to or at a tissue site. In some embodiments, the dressing 110 (or dressing assemblage) may be provided as a single unit.

The manifold 406 may include a first surface 414 and an opposing second surface 412. In some examples, at least a portion of the first surface 414 (e.g. the tissue-facing surface) of the manifold 406 may be configured to face the tissue site (e.g. the area of tissue around the extremity) through the treatment aperture 408. In some examples, the attachment device 404 may be positioned on or at a portion of the first surface 414 of the manifold 406. In some examples, the manifold 406 may include or be formed of a porous material, such as foam.

In some examples, the attachment device 404 may be configured to create a sealed space between the cover 140 and the tissue site, and the manifold 406 may be configured to be positioned in the sealed space. For example, the attachment device 404 may be positioned around an edge 416 of the manifold 406 and configured to surround the tissue site. The cover 140 may be disposed over the manifold 406 and coupled to the attachment device 404 around the manifold 406. For example, the cover 140 may be coupled to a portion of the attachment device 404 extending outward from the edge 416 of the manifold 406. Further, the cover 140 may be larger than the manifold 406, as illustrated in the example of FIG. 4, and may have a perimeter or a flange 418 configured to be attached to the attachment device 404. Assembled, the cover 140 may be disposed over the second surface 412 (e.g. the outward-facing surface) of the manifold 406, and the flange 418 may be attached to the attachment device 404 around the manifold 406. For example, an adhesive may be used to adhere the flange 418 to the attachment device 404, or the flange 418 may be, without limitation, welded, stitched, or stapled to the attachment device 404. In some embodiments, the attachment device may comprise an adhesive applied to the flange 418 and configured to allow attachment of the flange 418 to the tissue site and/or to a drape on the tissue site. The cover 140 may also include a port 420 configured to allow fluid communication between the manifold 404 and a dressing interface 422 and/or a fluid conductor 424 (e.g. to apply negative pressure under the cover) as described herein.

The attachment device 404 may take many forms. In some examples, the attachment device 404 may include or be formed of a film or membrane that can provide a seal in a therapeutic negative-pressure environment. In some example embodiments, the attachment device 404 may be a polymer film, such as a polyurethane film, that is permeable to water vapor but impermeable to liquid. The attachment device 404 may have a thickness in the range of 25-50 microns. For permeable materials, the permeability may be low enough that a desired reduced pressure may be maintained. The attachment device 404 may also include a medically-acceptable adhesive, such as a pressure-sensitive adhesive. In examples, the attachment device 404 may be a polymer film coated with an adhesive, such as an acrylic adhesive, which may have a coating weight between 25-65 grams per square meter (g.s.m.). Thicker adhesives, or combinations of adhesives, may be applied in some examples to improve the seal and reduce leaks.

In some examples, the attachment device 404 may include or be formed of a hydrocolloid. In some examples, the attachment device 404 may be configured or referred to as a sealing ring or a gasket member. In other examples, the dressing 110 may include a gasket member (not shown) in addition to the attachment device 404. In such an example, the gasket member may be a peripheral member, such as a hydrocolloid ring, and at least a portion of the attachment device 404 may be positioned between the manifold 406 and the gasket member on or at a surface of the manifold 406, such as the first surface 414, configured to face the area of tissue around the tissue site. In some examples, the gasket member may have a similar or analogous shape as the adhesive ring 410, but the gasket member may be positioned on a surface of the attachment device 404 configured to face the tissue site such that the gasket member is configured to be positioned between the tissue site and the attachment device 404.

In some examples, the dressing 110 may optionally further include a protective layer 425, which may be coupled to a surface of the manifold 406, such as the first surface 414, and may be configured to be exposed to the tissue site. In some embodiments, the protective layer 425 may be configured to be positioned in direct contact with the tissue site, for example forming a tissue-contact surface. In other embodiments (e.g. without a protective layer), the tissue-contact surface may be formed by the manifold and/or the attachment device. The protective layer 425 may include or be formed of a material that substantially reduces or eliminates skin irritation while allowing fluid transfer through the protective layer. In some embodiments, the protective layer 425 may form a fluid control layer, configured to allow fluid communication between the tissue site and the manifold during negative-pressure therapy, while minimizing backflow of fluids (such as exudate) from the manifold to the tissue site (e.g. to minimize maceration). In some examples, the protective layer 425 may include or be formed of one or more of the following materials, without limitation: a woven material, a non-woven material, a polyester knit material, and a fenestrated film.

In some examples, the attachment device 404, which may comprise an adhesive on a surface of the dressing 110 configured to face the tissue site (e.g. on the tissue-contact surface), may be covered by one or more release liners 428 prior to applying the dressing 110 at the tissue site. For example, as shown in FIG. 4, the dressing 110 may include a first release liner 428a, a second release liner 428b, and a third release liner 428c. The first release liner 428a may be positioned proximate to a first side 430 of the manifold 406 or the dressing 110, the second release liner 428b may be positioned proximate to a second side 432 of the manifold 406 or the dressing 110 (e.g. with the first side 430 and the second side 432 opposite each other across a line of symmetry 434), and the third release liner 428c may be positioned proximate to a fold axis, centerline, or line or symmetry 434 of the manifold 406 or the dressing 110 (e.g. spanning a central portion of the manifold and/or dressing). The central portion with the line of symmetry 434 may be located between the first side 430 and the second side 432, and the third release liner 428c may be positioned between the first release liner 428a and the second release liner 428b. In some examples, the third release liner 428c may be configured to be removed to expose an adhesive or portion of the attachment device 404 proximate to the line of symmetry prior to removal of the first release liner 428a and the second release liner 428b. Such a configuration may permit the central portion of the dressing 110 (e.g. in proximity to the line of symmetry 434) to be initially positioned or aligned at a tissue site, such as the extremity, while the first release liner 428a and the second release liner 428b protect other portions of the adhesive or the attachment device 404. For example, a portion of the third release liner 428c may cover or be positioned over a portion of the first release liner 428a and/or the second release liner 428b such that the third release liner 428c may be removed prior to removal of the first release liner 428a and the second release liner 428b. In some examples, the dressing 110 may have two release liners, each of which may have perforations or slits (not shown here) configured to allow the release liners to be separated into smaller pieces for removal. Additionally, some embodiments may also have one or more casting sheet liners 436.

Additionally or alternatively, the first release liner 428a, the second release liner 428b, and the third release liner 428c may provide stiffness to the attachment device 404 to facilitate handling and application. Additionally or alternatively, the casting sheet liners 436 may cover the flange 418 to provide stiffness to the cover 140 for handling and application. The one or more release liner 428 may be configured to releasably cover the attachment device 404, for example to protect and maintain the adhesive of the attachment device 404 until the time of application of the dressing 110 to the tissue site.

In some embodiments, the dressing 110 may be similar to that of FIG. 4, but may have a different shape. For example, the shape of the dressing may vary depending on the specific tissue site, wound cavity, and/or treatment for which the dressing may be used. In some embodiments, the dressing 110 may be shaped to include one or more arms or wings, for example extending from a stem or a central portion. FIG. 5 illustrates such an example dressing 110, which may have layers similar to those in FIG. 4, but may have a differently shaped perimeter.

FIG. 5 is a top view of another example of the dressing 110 that may be associated with an example embodiment of the therapy system of FIG. 1, illustrating additional details that may be associated with some embodiments. In the example embodiment of FIG. 5, the dressing 110 may include features that can cover articulating joints, such as a knee, while still allowing for significant range of motion. For example, the dressing 110 of FIG. 5 may generally comprise a manifold 406 having a stem 510, a first arm 515 joined to a first end of the stem 510, and a second arm 520 joined to a second end of the stem 510.

In some embodiments, the manifold 406 may be characterized as a polyhedron or as a generalized cylinder. For example, in FIG. 5 the manifold 406 can be characterized as a generalized cylinder having the second surface 412 and an edge 416. The edge 416 in FIG. 5 bounds the stem 510, the first arm 515, and the second arm 520. In some embodiments, some portions of the edge 416 may be curved, and some portions may be straight. In FIG. 5, for example, the first arm 515 is bounded in part by a first edge portion 535 that is substantially straight, and the second arm 520 is bounded in part by a second edge portion 540 that is substantially straight. In other embodiments, the first arm 515, the second arm 520, or both may be contoured at the extremities.

The stem 510 is generally configured to be positioned over an articular surface. The width of the stem 510 may vary for different types of joints, and may be limited to minimize interference with articulation. For example, in some embodiments, the stem 510 may be configured for positioning over a patella and have a width of 2-4 inches. In other examples, a width of 1-3 inches may be suitable for positioning over an olecranon.

As illustrated in the example of FIG. 5, the first arm 515 and the second arm 520 may flare away from the stem 510. In some embodiments, the width of the first arm 515 and the second arm 520 may each be greater than the width of the stem 510. In some examples, the face of the second surface 412 may be biconcave. More generally, portions of the edge 416 bounding the first arm 515 and the second arm 520 may converge toward the stem 510 to define a concave void adjacent to each side of the stem 510. In the example of FIG. 5, the concave void is curved. In other examples, the edge 416 may have straight segments that converge toward a vertex at the stem 510.

Some embodiments of the manifold 406 may additionally be characterized by a line of symmetry 434 (which may be a longitudinal centerline, in some examples) through the stem 510, and each of the first arm 515 and the second arm 520 may be characterized by a span that is generally orthogonal to the line of symmetry 434. In the example of FIG. 5, a first span 550 between extremities 555 is characteristic of the first arm 515, and a second span 560 between extremities 565 is characteristic of the second arm 520.

In the example of FIG. 5, the first span 550 is greater than the second span 560. A suitable ratio of the span of the first span 550 to the second span 560 may generally be in a range of 1.2 to 3.4. A ratio of 1.2 to 1.6 may be particularly advantageous for some applications. For example, in some embodiments the first span 550 may be in a range of 30-65 centimeters and the second span 560 may be in a range of 20-45 centimeters. In other examples, the first span 550 may be in a range of 15-50 centimeters and the second span 560 may be in a range of 8-25 centimeters.

In some embodiments, each arm (e.g. first arm 515 and second arm 520) may comprise two wing portions 580, one on each side of the line of symmetry 434 and extending outward away from the line of symmetry 434. In some embodiments, each of the wing portions 580 may extend away from the line of symmetry 434 in a direction substantially perpendicular to the line of symmetry 434. In FIG. 5, there are four wing portions: a first wing portion 580a, a second wing portion 580b, a third wing portion 580c, and a fourth wing portion 580d. For example, the first wing portion 580a and the second wing portion 580b may be symmetrical across the line of symmetry 434 and may jointly form the second arm 520; and the third wing portion 580c and the fourth wing portion 580d may be symmetrical across the line of symmetry 434 and may jointly form the first arm 515. In some embodiments, the first release liner 428a may cover the wing portions 580 on one side of the line of symmetry 434, and the second release liner 428b may cover the wing portions 580 on the other side of the line of symmetry 434. In some embodiments, a separate release liner may cover each wing portion 580 (which may allow the attachment device 404 on each wing portion 580 to be independently exposed), or a perforation line (not shown here) in the release liner 428 may allow exposure of the attachment device 404 on each wing portion 580 independently. In some embodiments, a central portion 582 of the manifold or dressing may extend along the line of symmetry 434, and the wing portions 580 may extend outward (e.g. substantially perpendicularly) from the central portion 582. The central portion 582 may, for example, span the center of the first arm 515, the second arm 520, and the stem 510. In some embodiments, the third release liner 428c may cover the attachment device on the central portion 582.

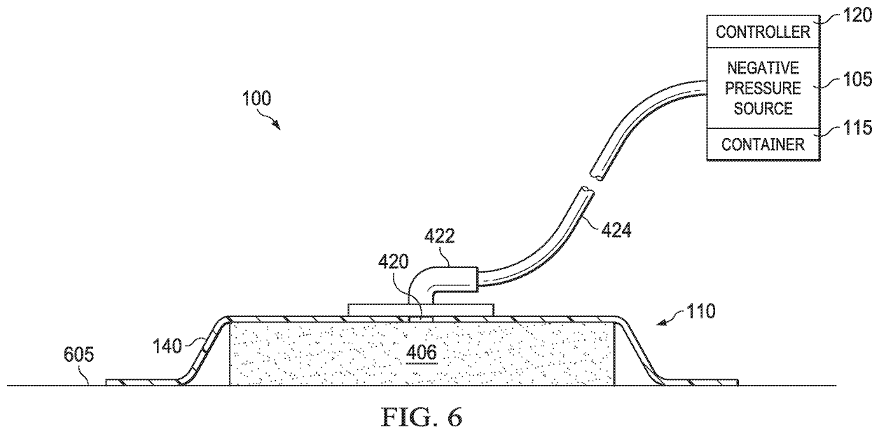

FIG. 6 is a schematic view illustrating an exemplary system 100 including a simplified example of the dressing 110 of FIG. 4 in place on an exemplary tissue site 605, illustrating additional details that may be associated with some embodiments. The system 100 may comprise a negative-pressure source 105 in fluid communication with the dressing 110. For example, the dressing 110 may comprise a dressing interface 422, which may penetrate or fluidly couple with the dressing 110 through the port 420 in the cover 140 to fluidly couple to the manifold 406 of the dressing 110. In some embodiments, a fluid conductor 424 may fluidly couple the negative-pressure source 105 to the dressing interface 422 (thereby fluidly coupling the negative-pressure source 105 to the manifold 406 of the dressing 110, for application of negative-pressure therapy to the tissue site 605 through the manifold 406 of the dressing 110).

In operation, the negative-pressure source 105 can reduce pressure in the sealed therapeutic environment (e.g. when the dressing 110 is applied to the tissue site 605 in the usage configuration). Reduced pressure applied to the tissue site 605 through the manifold 406 in the sealed therapeutic environment can induce macro-strain and/or micro-strain in the tissue site, as well as remove exudates and other fluids from the tissue site 605, which can be collected in the container 115.

In general, exudates and other fluids flow toward lower pressure along a fluid path. Thus, the term “downstream” may refer to a location in a fluid path relatively closer to a source of reduced pressure or further away from a source of positive pressure. Conversely, the term “upstream” may refer to a location further away from a source of reduced pressure or closer to a source of positive pressure.

In some example embodiments, the controller 120 may receive and process data from one or more sensors, such as the first sensor 125. The controller 120 may also control the operation of one or more components of the therapy system 100 to manage the pressure delivered to the tissue interface 135, such as the manifold 406 and associated components. In some embodiments, the controller 120 may include an input for receiving a desired target pressure, and may be programmed for processing data relating to the setting and inputting of the target pressure to be applied to the tissue interface 135. In some example embodiments, the target pressure may be a fixed pressure value set by an operator as the target reduced pressure desired for therapy at a tissue site and then provided as input to the controller 120. The target pressure may vary from tissue site to tissue site based on the type of tissue forming a tissue site, the type of injury or wound (if any), the medical condition of the patient, and the preference of the attending physician. After selecting a desired target pressure, the controller 120 can operate the negative-pressure source 105 in one or more control modes based on the target pressure, and may receive feedback from one or more sensors to maintain the target pressure at the tissue interface 135. In some embodiments, the manifold 406 may have distinct pressure zones, and different target pressures and control modes may be applied to different pressure zones.

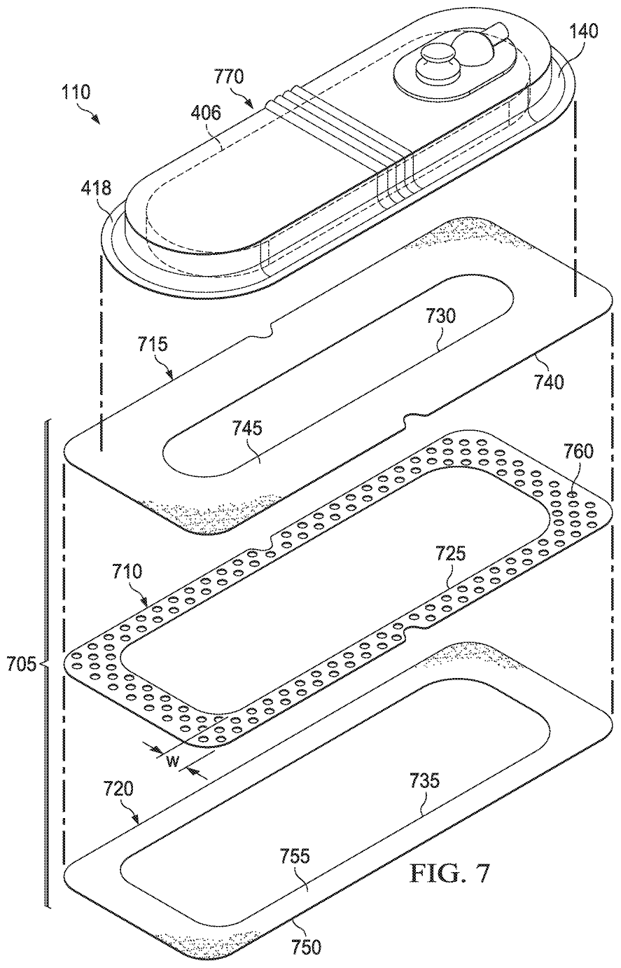

FIG. 7 is an exploded isometric view of another dressing that may be associated with an example embodiment of the therapy system of FIG. 1, illustrating additional details that may be associated with some embodiments. The dressing 110 in FIG. 7 may comprise a drape 705 having integral support. For example, the drape 705 may have an integral, semi-rigid support layer 710. The support layer 710 may be configured to provide sufficient support so that the drape 705 is self-supporting. For example, the drape 705 may be configured to be self-supporting prior to application of the dressing 110 to the tissue site, and may be configured to allow application of the drape 705 to the tissue site. In some embodiments, the support layer 710 may be sufficiently rigid to render the drape 705 self-supporting (e.g. not likely to fold over onto itself due to its own weight and/or sufficiently rigid so that the edges will not substantially droop, for example so that the edges hang down less than 90 degrees, less than 45 degrees, less than 20 degrees, or less than 10 degrees, under its own weight), but sufficiently flexible, pliable, conformable, and/or malleable to allow the drape 705 to be applied and close coupled to the tissue site (e.g. which may involve curvature). In some embodiments, the support layer 710 may be sufficiently rigid to maintain the dressing approximately within a flat plane under its own weight (e.g. so that additional force, for example applied by a user's hand, would have to be applied to bend the drape substantially out of the flat plane). In some embodiments, the support layer 710 may have a stiffness similar to (e.g. approximately the same as) or greater than (for example, by +5%, +10%, or +20%) that of 78 #casting paper. For example, the support layer 710 may comprise material having a Young's Modulus of approximately 0.1-10 GPa and a thickness of about 0.01 inch or less. In some embodiments, the Young's Modulus may be higher, for example up to 100 GPa, for a thinner support layer. In some embodiments, the support layer 710 may be configured so that, when the drape 705 is applied to the tissue site, substantially no forces are introduced to the tissue site due to elasticity of the support layer 710. In some embodiments, the support layer 710 may be configured to be left in place when the dressing or drape 705 is attached to the tissue site and in use, for example with the integral support layer 710 being permanently attached to and non-removable from the drape 705 (e.g. the support layer 710 cannot be removed from the drape 705 without significantly damaging the drape 705). In some embodiments, the support layer 710 may be configured within the drape 705 so as to not be visible (e.g. covered on all sides, so that an end-user cannot discern the support layer 710 merely by visual inspection of the drape 705).

In some embodiments, the drape 705 may be configured to seal the tissue site for negative-pressure therapy. For example, the drape 705 may comprise a material similar to the cover 140. In some embodiments, the support layer 710 may be embedded within the drape 705. For example, the support layer 710 may be internal to the drape 705. In some embodiments, the support layer 710 may be configured to support the drape 705 substantially around a perimeter of the drape 705, for example only directly providing support around the perimeter without directly supporting the central portion of the drape 705. For example, the support layer 710 may comprise an interior aperture 725, and in some embodiments the support layer 710 around the interior aperture 725 may span the perimeter of the drape 705 (e.g. be positioned around only the perimeter of the drape). In some embodiments, the interior aperture 725 may span a majority of the surface area of the support layer 710, leaving only a narrow perimeter border of support. In some embodiments, the perimeter of the drape 705 may be supported for a border width w of approximately ⅛-¾ inch (and within this approximately ⅛-¾ inch wide border around the perimeter, may lie the interior aperture 725).

In some embodiments, the dressing 110 may further comprise: an attachment device 404 having a treatment aperture 408; a manifold 406 configured to be in fluid communication with the tissue site through the treatment aperture 408 (e.g. disposed over the treatment aperture 408); and a cover 140 configured to be disposed over the manifold 406 and coupled to the attachment device 404 around the manifold 406. In some embodiments, the cover 140 may comprise a flange 418 extending beyond the manifold 406. In some embodiments, the attachment device 404, manifold 406, and/or cover 140 may be pre-assembled (e.g. coupled) to form a single unit, such as a dressing assemblage 770, which may be similar to the dressing shown in FIG. 4. In some embodiments, the drape 705 may be attached to the dressing assemblage 770, forming a single, integral dressing 110 unit. In some embodiments, the drape 705 may be separate but configured for use with the dressing assemblage 770, for example with the drape 705 configured to be applied to the tissue site, and the dressing assembly configured to be applied over the drape 705 in fluid communication with the tissue site through the drape 705 (e.g. through one or more apertures in the drape 705). In some embodiments, the interior aperture 725 of the support layer 710 may be larger than the treatment aperture of the dressing assemblage 770.

In some embodiments, the drape 705 may further comprise an outer drape layer 715 and a base drape layer 720, with the support layer 710 located therebetween. For example, the support layer 710 may be stacked between and in contact with the outer drape layer 715 and the base drape layer 720. In some embodiments, the outer drape layer 715 may comprise an interface aperture 730, the base drape layer 720 may comprise a base aperture 735, and the interface aperture 730 and the base aperture 735 may be vertically aligned with each other and with the interior aperture 725 of the support layer 710. For example, the interface aperture 730, the interior aperture 725, and the base aperture 735 may all be centered on a common central axis (e.g. extending orthogonal to the drape layers), may be co-axial, and/or may be concentric.

In some embodiments, the interface aperture 730 may be configured to interface with the treatment aperture 408 of the dressing assemblage 770. For example, the interface aperture 730 of the outer drape layer 715 may be configured to fluidly communicate with the treatment aperture 408 (and therethrough to the manifold 406). In some embodiments, the interface aperture 730 may be configured to fluidly communicate with the interior aperture 725 of the support layer 710, and thereby to the base aperture 735 (leading to the tissue site). In some embodiments, the interface aperture 730 may be configured to fluidly couple the treatment aperture 408 to the interior aperture 725, and the interior aperture 725 may be configured to fluidly couple the interface aperture 730 to the base aperture 735 (and thereby to the tissue site).

In some embodiments, the interface aperture 730 may be approximately the size of the treatment aperture 408. In some embodiments, the interface aperture 730 may be sized to be smaller than the flange 418 of the cover 140 (e.g. the flange 418 of the cover may extend beyond the interface aperture 730 when coupled). In some embodiments, the interface aperture 730 may be smaller than the interior aperture 725 of the support layer 710. In some embodiments, the base aperture 735 may be approximately the same size as the interior aperture 725 of the support layer 710. In the embodiments, the perimeter of the outer drape layer 715, the support layer 710, and the base drape layer 720 may all be approximately the same size and/or stacked in alignment (e.g. so that the outer edges of the drape layers all approximately match, align, and/or are flush).

In some embodiments, the support layer 710 may be stacked or sandwiched between and coupled to both the outer drape layer 715 and the base drape layer 720. For example, the support layer 710 may be adhered or otherwise bonded (e.g. heat bonded or welded) to both the outer drape layer 715 and the base drape layer 720. In some embodiments, the base drape layer 720 may be adhesive on a surface (e.g. a contact surface 750, configured to contact the tissue site) opposite the support layer 710. For example, the outer drape layer 715 may comprise a first surface 740, configured to face towards and/or contact the support layer 710, and a second surface 745 opposite the first surface 740, with the first surface 740 being adhesive (e.g. the first surface 740 of the outer drape layer 715 may comprise a first adhesive) and the second surface 745 of the outer drape layer 715 not being adhesive. In some embodiments, the base drape layer 720 may comprise a contact surface 750 configured to contact the tissue site and an exterior surface 755 opposite the contact surface 750 (e.g. configured to contact the support layer 710), with both the contact surface 750 and the exterior surface 755 being adhesive (e.g. the contact surface 750 may comprise a second adhesive and the exterior surface 755 may comprise a third adhesive). In some embodiments, the second adhesive (e.g. on the contact surface 750) may be configured to sealing and removably attach the dressing 110 or drape 705 to the tissue site. For example, the second adhesive may comprise or consist essentially of one or more of the following: acrylic and hydrocolloid. In some embodiments, the first adhesive and the third adhesive (e.g. the adhesives contacting the support layer 710) may be configured to securely and/or permanently attach the drape layers to the support layer 710. For example, the first and third adhesives may each comprise or consist essentially of one or more of the following: acrylate, acrylate double-sided adhesive, and 3M™ Double Coated Medical Tape Product Number 1522. In some embodiments, the first adhesive and the third adhesive may be the same, while in other embodiments they may differ.

In some embodiments, substantially the entire base drape layer 720 may be adhered to substantially the entire support layer 710. In some embodiments, the base drape layer 720 may be approximately the same size as the support layer 710. In some embodiments, only a perimeter of the outer drape layer 715 may be adhered to the support layer 710 (e.g. an interior portion of the outer drape layer 715 may not contact or adhere to the support layer 710). In some embodiments, the support layer 710 and/or the base drape layer 720 may support the perimeter of the drape 705 by extending in from the perimeter for a border width w of approximately ⅛-¾ inch. In some embodiments, the border width w may be approximately uniform about the entire perimeter, while in other embodiments, the border width w may vary within the range of approximately ⅛-¾ inch along the perimeter. In some embodiments, the outer drape layer 715, the support layer 710, and the base drape layer 720 may be pre-formed as an integral drape 705 unit (with integral support).

In some embodiments, the outer drape layer 715 and/or the base drape layer 720 may be similar to the cover 140 in FIG. 4, for example comprising or consisting essentially of similar materials and/or having similar thickness. For example, the outer drape layer 715 and/or the base drape layer 720 may comprise or consist essentially of a polymer film, such as one or more of the following: polyurethane, such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; polyether block polyamide copolymer (PEBAX); or other appropriate material. In some embodiments, the outer drape layer 715 and/or the base drape layer 720 may have a thickness of approximately 25-50 microns, for example approximately 30 microns. In some embodiments, the outer drape layer 715 and/or the base drape layer 720 may have a thickness of about 3 mil, about 3-5 mil, or less than about 5 mil. In some embodiments, the outer drape layer 715 and/or the base drape layer 720 may have a high MVTR, such as at least 250, at least 2600, at least 5000, 250-5000, 250-2600, or 2600-5000 grams per square meter per twenty-four hours.

In some embodiments, the support layer 710 may comprise a polymer film, which may be thicker and/or stiffer (e.g. formed of a stiffer material) than the outer drape layer 715 and/or the base drape layer 720. For example, the support layer 710 may comprise or consist essentially of a polyurethane film, which may be approximately 0.01 inch thick. In some embodiments, the support layer 710 may comprise or consist essentially of a polycarbonate film. In some embodiments, the support layer 710 may have a thickness that is approximately an order of magnitude greater than the thickness of the outer drape layer 715 and/or the base drape layer 720. In some embodiments, the support layer 710 may have a thickness of about 0.01 inch or less. In some embodiments, the support layer 710 may be configured to have an MVTR similar to the cover 140 and/or outer drape layer 715, and the similar MVTR may be either due to material property (e.g. porosity) of the support layer 710 or perforations 760 in the support layer 710 configured to provide the MVTR. For example, if the polymer film material forming the support layer 710 has a low MVTR (or a lower MVTR than the outer drape layer 715), the support layer 710 may comprise a plurality of perforations 760 in the film material which may be configured to provide MVTR similar to the cover 140 and/or outer drape layer 715. For example, each of the perforations 760 may have a diameter of approximately 2-5 mm, and the total exposed area (e.g. area of the support layer without support material) may be approximately 20-40%.

In some embodiments, the support layer 710 may comprise or consist essentially of a mesh, such as a semi-rigid mesh. In some embodiments, the mesh of the support layer 710 may be formed by interwoven strands of material, such as a polymer (e.g. PU or polycarbonate), a metal wire, or carbon fiber. In some embodiments, the mesh of the support layer 710 may be formed by a plurality of perforations 760 in a polymer sheet or film. In some embodiments, the support layer 710 may comprise a polymer film embedded with carbon fibers. In some embodiments, the carbon fibers may form a mesh (e.g. located within the polymer film). For example, the carbon fibers may be formed into a fabric (e.g. woven) sheet that is located within the film, and the fabric may be located in proximity to a central plane of film. In some embodiments, the mesh of carbon fiber strands may comprise weft and warp strands (e.g. a grid) that are approximately orthogonal. In some embodiments, the support layer 710 may comprise a PU film with carbon fibers. In some embodiments, the support layer 710 may comprise a polycarbonate film with carbon fibers. In some embodiments, the carbon fibers may form approximately 2-5% of the support layer 710. In some embodiments, the support layer 710 may comprise or consist essentially of a skeleton support structure, for example having a plurality of supports coupled together as discussed with respect to embodiments below.

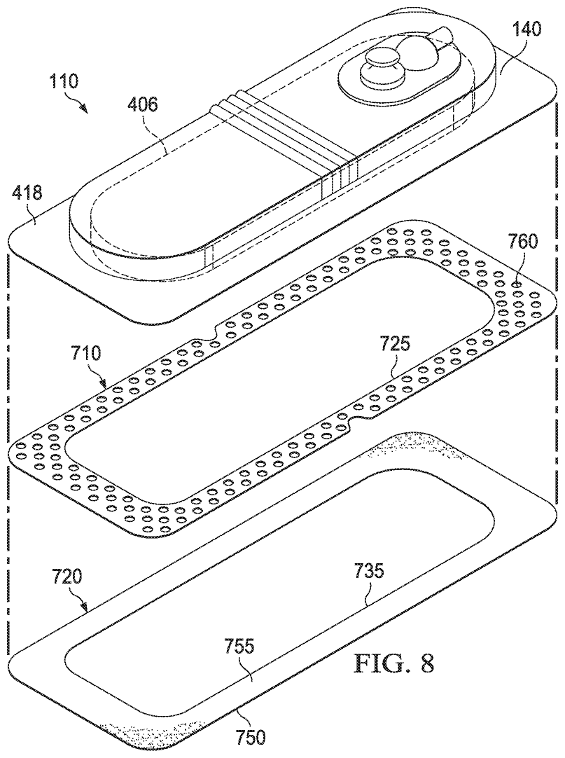

FIG. 8 is an exploded isometric view of yet another dressing that may be associated with an example embodiment of the therapy system of FIG. 1, illustrating additional details that may be associated with some embodiments. In some embodiments, the outer drape layer or the drape (for example, as described in FIG. 7) as a whole may be or may serve as the cover 140 (e.g. for a dressing 110). For example, the outer drape layer 715 or the drape 705 as a whole may form a solid sheet or film without any aperture or opening, which is configured to seal a tissue site for negative-pressure therapy. For example, such an outer drape layer 715 may be configured for use over a manifold 406. In some embodiments, a dressing interface 422 may be coupled to the second surface 745 of the outer drape layer 715, for example to provide negative pressure to a manifold 406 located thereunder.

The embodiment shown in FIG. 8 may be similar to the embodiment shown in FIG. 7, except that the cover 140 may form and/or comprise the outer drape layer 715 (e.g. the outer drape layer of the drape may form the cover 140). For example, a portion of the cover 140 (e.g. the portion of the cover 140 not disposed over the manifold 406 and/or which extends beyond the manifold 406) may form the outer drape layer 715. In some embodiments, the cover 140 may comprise the flange 418 extending outward beyond a perimeter of the manifold 406, and the flange 418 of the cover 140 may form the outer drape layer 715. In some embodiments, the flange 418 may couple to the attachment device for the dressing 110. In some embodiments, a separate attachment device 404 may not be necessary. For example, the drape may function as the attachment device 404 (e.g. for attaching the manifold 406 to the tissue site and allowing fluid communication between the manifold 406 and the tissue site). By way of example, the adhesive on the contact surface 750 of the base drape layer 720 may be configured to attach the dressing to the tissue site (e.g. serving as the attachment device 404). In some embodiments, the base drape layer 720 may form the attachment device.

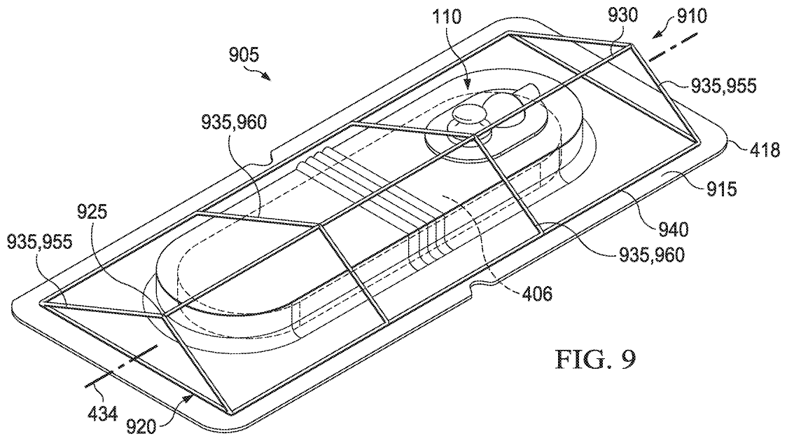

In some embodiments, such as in FIGS. 9-12, the support layer may be externally coupled to the cover 140 of the dressing 110. In some embodiments, the support layer may be coupled to or on (e.g. directly coupled to) an outer surface 915 of the cover 140, for example the outer surface 915 of the flange 418 of the cover 140 extending laterally beyond the manifold 406 of the dressing 110. In some embodiments, the support layer may render the flange 418 of the cover 140 self-supporting. In some embodiments, the support layer may be removably coupled to the cover 140, so that it may assist in preventing folding over of the cover 140 during placement of the dressing 110, and may be removed from the dressing 110 after the dressing 110 has been placed on the tissue site. In some embodiments, the support layer may be planar and may be permanently attached to the cover 140 (e.g. the outer surface 915 of the flange 418). In some embodiments, an upper film layer or outer drape layer 715 may be disposed over the support layer. For example, the support layer may be externally adhered to the outer surface 915 of the cover 140, with the support layer disposed between the cover 140 and the upper film layer. In some embodiments, the support layer may comprise a skeleton support structure 910. In some embodiments, the support layer, such as the skeleton support structure 910, may be non-planar, for example extending from the outer surface 915 of the flange 418 over the manifold 406. FIGS. 9-12 illustrate in more detail related exemplary embodiments.

FIG. 9 is an isometric view of an example embodiment of a dressing assembly 905, having a dressing 110 similar to that of FIG. 4 and the skeleton support structure 910, that may be associated with the therapy system of FIG. 1 and may illustrate additional details that may be associated with some embodiments. The dressing 110 of FIG. 9 comprises the manifold 406 and the cover 140 disposed over the manifold 406 (e.g. over the second surface 412 of the manifold 406). In some embodiments, the cover 140 may comprise the flange 418 extending laterally beyond the manifold 406, in proximity to (e.g. in substantially the same plane as) the first surface 414 of the manifold 406. The skeleton support structure 910 of FIG. 9 is coupled to an outer surface 915 of the flange 418 and is configured to support the flange 418. In some embodiments, the skeleton support structure 910 may be configured to render the flange 418 self-supporting. For example, the skeleton support structure may be sufficiently rigid to render the flange 418 self-supporting (e.g. not likely to fold over onto itself due to its own weight) prior to application of the dressing 110 to the tissue site, and may be configured to allow application of the dressing 110 to the tissue site (e.g. sufficiently flexible, pliable, conformable, and/or malleable to allow the flange 418 to be applied and close coupled to the tissue site, which may involve curvature). In some embodiments, the skeleton support structure 910 may be semi-rigid. For example, the skeleton support structure 910 may be sufficiently rigid to maintain the flange 418 approximately within a flat plane under its own weight. In some embodiments, the skeleton support structure 910 may have a stiffness approximately the same as or greater than that of 78 #casting paper. In some embodiments, the skeleton support structure 910 (e.g. the supports forming the skeleton support structure 910) may have a Young's Modulus of about 1-10 GPa. In some embodiments, the skeleton support structure 910 may be configured to prevent the flange 418 from folding over onto itself, and may be sufficiently malleable or flexible to allow positioning of the dressing 110 on the tissue site.

In some embodiments, the skeleton support structure 910 or other support layer may be similar to the support layer 710 in FIG. 7 (for example, having similar properties or being formed of similar materials). In some embodiments, the skeleton support structure 910 may be coupled to and configured to support a perimeter of the flange 418. In some embodiments, the skeleton support structure 910 may be coupled to a casting sheet liner, similar to that of FIG. 4, which may be coupled to the outer surface 915 of the flange 418. In some embodiments, the casting sheet liner may be located between the flange 418 and the skeleton support structure 910. Some embodiments may comprise the attachment device 404 with one or more release liners 428. In some embodiments, the flange 418 may be located between the one or more release liners 428 and the skeleton support structure 910.

In some embodiments, the skeleton support structure 910 may be non-planar, as shown in FIG. 9. For example, the skeleton support structure 910 may extend from the outer surface 915 of the flange 418, outward above the outer surface 915 of the flange 418. In some embodiments, the skeleton support structure 910 may extend outward from the outer surface 915 of the flange 418, beyond and/or over the manifold 406 (for example, extending outward from the outer surface 915 of the flange 418 for a height no less than the thickness of the manifold 406). In some embodiments, the skeleton support structure 910 may comprise a base portion 920 configured to support the flange 418, and a projection portion 925 configured to extend vertically above the flange 418 and/or over the manifold 406. In some embodiments, the projection portion 925 may extend at least about % inch above the manifold 406 (e.g. above the second surface 412 of the manifold 406), such that there may be a space between the manifold 406 and the projection portion 925 above the manifold 406 which is at least about % inch. The base portion 920 may be coupled to the projection portion 925, to form an integral skeleton support structure 910. In some embodiments, the projection portion 925 may form a handle, allowing handling of the dressing 110 during placement while the base portion 920 supports the flange 418. The space between the manifold 406 and the projection portion 925 may allow a user to grasp the projection portion 925 effectively, for example with one or more fingers between the projection portion 925 and the manifold 406.

In some embodiments, the skeleton support structure 910 may be removably coupled to the outer surface 915 of the flange 418. For example, the base portion 920 may be removably coupled to the outer surface 915 of the flange 418. Removable coupling may allow the skeleton support structure 910 to support the flange 418 during placement of the dressing 110 on the tissue site (e.g. preventing the cover/flange 418 from folding over), while allowing the skeleton support structure 910 to be removed after the dressing 110 has been placed on the tissue site. The removable coupling of the skeleton support structure 910 to the flange 418 is configured so that removal of the skeleton support structure 910 from the flange 418 does not substantially damage the cover 140. In some embodiments, the skeleton support structure 910 may be removably attached to the outer surface 915 of the flange 418 by a low-tack adhesive. For example, the base portion 920 of the skeleton support structure 910 may comprise the low-tack adhesive. In some embodiments, the low-tack adhesive may have an adhesive strength from about 0.2N to about 16.1N. In some embodiments, the low-tack adhesive may comprise low-tack acrylic adhesive. In some embodiments, the dressing 110 may further comprise an adhesive attachment device 404 (e.g. on the inner surface of the cover 140, as shown in FIG. 4) configured to couple and seal the dressing 110 to the tissue site for negative-pressure treatment. In some embodiments, the low-tack adhesive removably coupling the skeleton support structure 910 to the outer surface 915 of the flange 418 may have a lower adhesion strength than the adhesive attachment device 404 for the dressing 110. Such relative adhesion strength may allow the skeleton support structure 910 to be removed from the flange 418 after the dressing 110 has been placed on the tissue site, without substantially impacting the adhesion and/or seal of the dressing 110 to the tissue site. In some embodiments, the skeleton support structure 910 (e.g. the base portion 920) may be removably coupled to the outer surface 915 of the flange 418 by static cling or other removable attachment means.

In some embodiments, the skeleton support structure 910 may have an I-beam shape. In some embodiments, the skeleton support structure 910 may comprise a plurality of supports (e.g. elongate support members) coupled together into a unitary whole. In some embodiments, the skeleton support structure 910 may comprise a longitudinal support 930, which may extend approximately parallel to the line of symmetry 434 or centerline of the dressing 110, and at least two lateral supports 935, which may extend from the longitudinal support 930 to substantially the perimeter of the flange 418. The at least two lateral supports 935 may be coupled to the longitudinal support 930. For example, the at least two lateral supports 935 may couple the longitudinal support 930 to the flange 418 (e.g. in proximity to the perimeter of the flange 418). In some embodiments, the lateral supports 935 may not contact or be coupled to the dressing 110 elsewhere (e.g. only contacting the perimeter of the flange 418). In some embodiments, the longitudinal support 930 may be substantially located on the line of symmetry 434 or centerline of the dressing 110 (e.g. over the manifold 406). In some embodiments, the longitudinal support 930 may extend over at least substantially the length of the manifold 406 or the dressing 110. In some embodiments, the lateral supports 935 may each extend substantially the width of the flange 418 or dressing 110. In some embodiments, the lateral supports 935 may each extend from opposite sides of the longitudinal support 930 to substantially the perimeter of the flange 418, substantially spanning the width of the dressing 110. For example, each of the lateral supports 935 may couple the longitudinal support 930 to the flange 418 on opposite sides of the manifold 406. In some embodiments, each of the lateral supports 935 may comprise two lateral support elements, which may symmetrically couple the longitudinal support 930 to the flange 418.

In some embodiments, the at least two lateral supports 935 may comprise two end lateral supports 955. In some embodiments, the end lateral supports 955 may be coupled to the longitudinal support 930 in proximity to opposite ends of the longitudinal support 930, forming the I-beam shape of the skeleton support structure 910. In some embodiments, the projection portion 925 may comprise the longitudinal support 930, which may be disposed over and above the manifold 406. In some embodiments, the lateral supports 935 may further comprise one or more intermediate lateral supports 960 located between the two end lateral supports 955 and coupling the longitudinal support 930 to the flange 418. In some embodiments, the lateral supports 935 may be evenly spaced. In some embodiments, the spacing between lateral supports 935 may be about 2-3 inches. In some embodiments, the lateral supports 935, for example the end lateral supports 955, may couple the longitudinal support 930 to the flange 418 in proximity to corners of the flange 418.

In some embodiments, the base portion 920 may comprise one or more perimeter supports 940, configured to support the flange 418 around its perimeter (e.g. extending along the outer circumference of the flange 418 and/or contacting the flange 418 along the entire length of the perimeter supports 940). In some embodiments, the lateral supports 935 may couple the base portion 920 to the projection portion 925. For example, the lateral supports 935 may couple the perimeter supports 940 to the longitudinal support 930, holding the longitudinal support 930 above the manifold 406. In some embodiments, the skeleton support structure 910 may have a peaked shape, for example forming a triangular tent-like frame extending along the line of symmetry 434 or centerline of the dressing 110 or manifold 406. In some embodiments, the lateral supports 935 may extend approximately perpendicularly from the longitudinal support 930. In some embodiments, the skeleton support structure 910 may be approximately symmetrical about the line of symmetry 434 or centerline of the manifold 406 or dressing 110. In some embodiments, the skeleton support structure 910 may be external to the dressing 110, for example externally attached to the outer surface 915 of the flange 418.

In some embodiments, each of the supports may be semi-rigid. For example, each of the supports may have a Young's Modulus of about 1-10 GPa. In some embodiments, the skeleton support structure 910 may comprise and/or be formed of wire. In some embodiments, each of the supports (e.g. the longitudinal support 930 and the lateral supports 935) of the skeleton support structure 910 may comprise wire, which may be similar to that used for pipe cleaners (e.g. chenille stems) or twist-ties. For example, each of the supports of the skeleton support structure 910 may comprise coated metal filament wire. In some embodiment, the wire may comprise 1-1.5 mm coated copper or steel wire, which may be coated with polyvinyl chloride (PVC) or PU. In some embodiments, each of the supports may comprise a thin molded component, for example formed of a polymer such as polypropylene (PP), acrylonitrile butadiene styrene (ABS), or polyethylene terephthalate glycol (PETG), with a diameter or thickness of about 0.020-0.200 inch. In some embodiments, the longitudinal support 930 may be thicker and/or more rigid than the lateral supports 935.

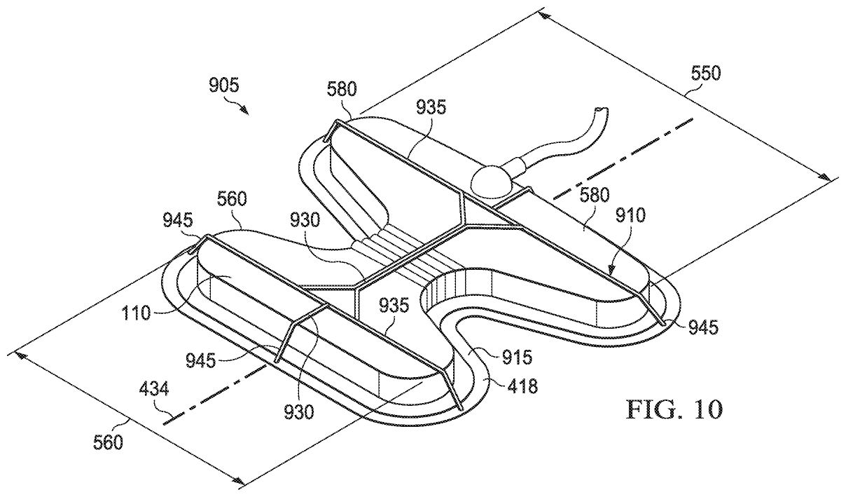

FIG. 10 is an isometric view of another example embodiment of a dressing assembly 905, similar to that of FIG. 9 but having a different shape and illustrating additional details that may be associated with some embodiments. In FIG. 9, the dressing 110 is shaped similar to that shown in FIG. 5. For example, the dressing 110 may comprise one or more wing portions 580 extending laterally away from the line of symmetry 434 or longitudinal centerline. In some embodiments, two wing portions 580 may be symmetrically located across the line of symmetry 434, jointly forming an arm. In some embodiments, for each wing portion 580 or arm, one of the lateral supports 935 may span the wing portion 580 or arm to couple the longitudinal support 930 to the flange 418 (e.g. in proximity to the perimeter of the wing portion 580 or arm). In some embodiments, the lateral supports 935 may couple the longitudinal support 930 to the flange 418 in proximity to the widest portions of the flange 418. For example, one of the lateral supports 935 may extend across the first span 550, and one of the lateral supports 935 may extend across the second span 560.

In some embodiments, the longitudinal support 930 may couple to the flange 418 at opposite ends, for example in proximity to the line of symmetry 434 or longitudinal centerline. In some embodiments, the longitudinal support 930 may comprise a plurality of longitudinal elements coupled together to form an integral longitudinal support 930. In some embodiments, the longitudinal elements may be coupled together via attachment to one or more lateral supports 935. In some embodiments, one or more of the longitudinal elements may not be parallel to the line of symmetry 434 or longitudinal centerline, but overall and taken as a whole, the longitudinal support 930 may extend substantially along the line of symmetry 434 or longitudinal centerline and/or may be configured to support the dressing 110 longitudinally. For example, in FIG. 10 some of the longitudinal elements may be diagonal to the line of symmetry 434 or longitudinal centerline.

In some embodiments, the base portion of the skeleton support structure 910 may comprise or consist essentially of the portions of the lateral supports 935 and/or the longitudinal support 930 contacting the flange 418. In some embodiments, the projection portion of the skeleton support structure 910 may comprise or consist essentially of the portions of the lateral supports 935 and/or longitudinal support 930 spanning the manifold 406 and/or projecting above the outer surface 915 of the flange 418. In some embodiments, the lateral supports 935 and/or the longitudinal support 930 may each comprise one or more (e.g. two, with one located at each end) tip 945 configured to contact the drape or cover in proximity to the perimeter (e.g. with the one or more tip 945 forming the portion of the skeleton support structure 910 contacting the flange 418). For example, each tip 945 may be joined to the projection portion of the corresponding lateral support 935 or longitudinal support 930 by a bend. In some embodiments, the base portion of the skeleton support structure 910 may comprise or consist essentially of all of these tips 945 as a collective. In some embodiments, the projection portion (e.g. the portion of the longitudinal support 930 and/or the lateral supports 935 extending over the manifold 406) may be substantially planar and located in a plane above and/or over the manifold 406 (e.g. spaced about % inch above the manifold 406). In some embodiments, the tips 945 may each bend downward from the projection portion to contact the flange 418. In some embodiments, the tips 945 may jointly hold the projection portion above and/or over the manifold 406.

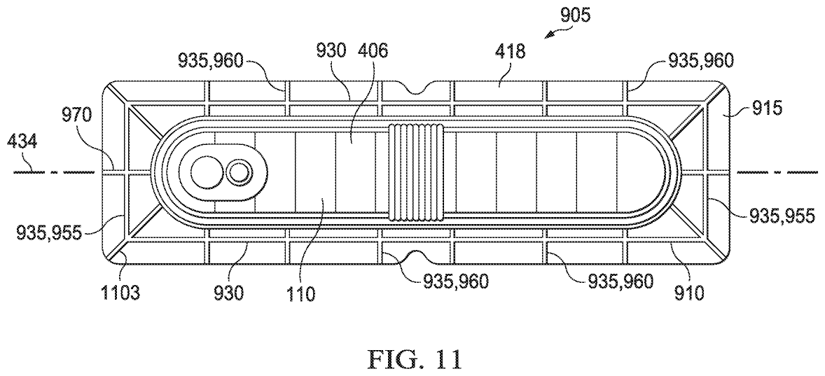

FIG. 11 is a top plan view of yet another example embodiment of the dressing assembly 905 that may be associated with an example embodiment of the therapy system of FIG. 1, illustrating additional details that may be associated with some embodiments. In some embodiments, the support layer may be substantially planar, and may be disposed on and coupled to the outer surface 915 of the flange 418 of the dressing 110. For example, the support layer may be similar to that described with respect to FIGS. 9-10 (e.g. a skeleton support structure comprising supports), but may be planar in shape. In other embodiments, the support layer may be similar to that of FIG. 7, but may be disposed on the outer surface 915 of the flange 418 of the cover 140 or the drape.

The embodiment of FIG. 11 comprises a substantially planar (e.g. 2D) skeleton support structure 910 which is disposed on and coupled to the outer surface 915 of the flange 418 of the dressing 110. In some embodiments, the skeleton support structure 910 may not extend over the manifold 406. For example, in FIG. 11 the skeleton support structure 910 may only comprise the base portion, and may not include any projection portion. In some embodiments, the planar skeleton support structure 910 may extend laterally around the manifold 406 in a plane. For example, all of the supports forming the skeleton support structure 910 may be disposed in a plane atop the outer surface 915 of the flange 418. In some embodiments, the supports may be disposed on the outer surface 915 of the flange 418, and in a plane around the manifold 406. In some embodiments, the supports of the skeleton support structure 910 may support the flange 418 around the manifold 406, for example being configured to hold the flange 418 substantially in a plane with the outer surface 915 of the first surface of the manifold 406. In some embodiments, the skeleton support structure 910 may be removably coupled to the flange 418. In other embodiments, the skeleton support structure 910 may be permanently attached to the flange 418.

In some embodiments, the skeleton support structure 910 may comprise at least two longitudinal supports 930, for example extending substantially parallel to the line of symmetry 434 and/or longitudinal centerline. In some embodiments, the two longitudinal supports 930 may be symmetrically disposed on opposite sides of the manifold 406 (e.g. symmetrical across the line of symmetry 434), for example with one of the at least two longitudinal supports 930 disposed on each side of the manifold 406. The plurality of lateral supports 935 may extend from proximity to the manifold 406 to proximity to the perimeter edge of the flange 418 on both sides of the manifold 406. In some embodiments, the lateral supports 935 may extend symmetrically across the line of symmetry 434. In some embodiments, the skeleton support structure 910 may further comprise a plurality of corner supports 1103, which may be configured to extend from proximity to the manifold 406 to the perimeter of the flange 418 at a corner of the flange 418. In some embodiments, the plurality of lateral supports 935 may comprise two end lateral supports 955. In some embodiments, the end lateral supports may extend between and couple the two longitudinal supports 930. For example, each of the end lateral supports may be coupled to ends of the two longitudinal supports 930 across the line of symmetry 434 (e.g. with the end lateral supports and the two longitudinal supports 930 jointly surrounding the manifold 406 symmetrically in the plane of the outer surface 915 of the flange 418). In some embodiments, the plurality of lateral supports 935 may further comprise intermediate lateral supports 960, which may be evenly spaced between the end lateral supports 955. In some embodiments, the two or more longitudinal supports 930 may further comprise a centerline longitudinal support 970, which may extend approximately along the line of symmetry 434 or centerline of the manifold 406 or dressing 110. In some embodiments, the centerline longitudinal support 970 may comprise two longitudinal elements, for example each extending from opposite ends of the manifold 406 to the perimeter of the drape. In some embodiments, each of the intermediate lateral supports 960 may comprise two lateral elements, symmetrically disposed across the line of symmetry 434. In some embodiments, each lateral element of an intermediate lateral support 960 may extend from the manifold to the perimeter of the flange 418.

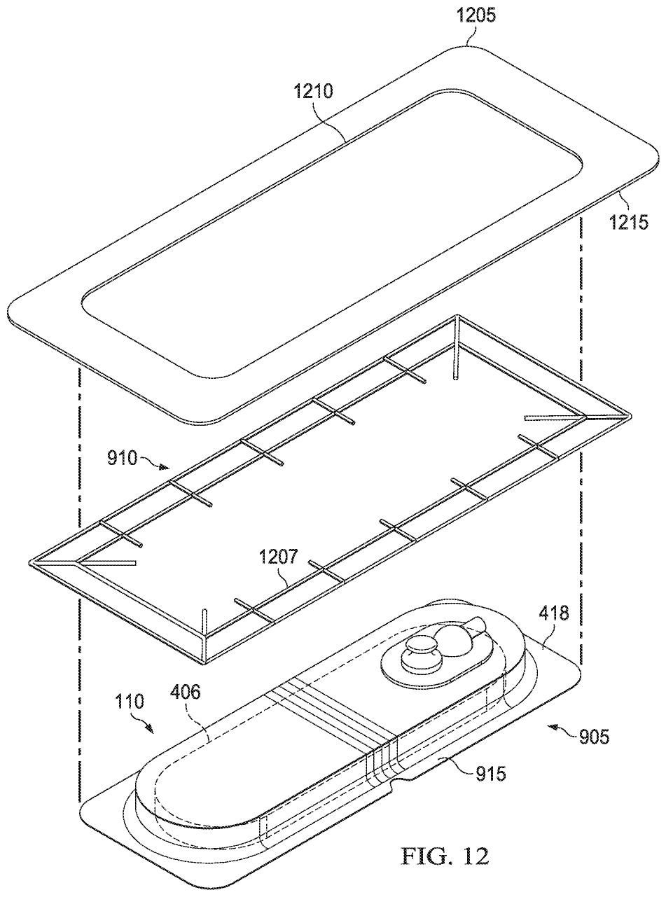

FIG. 12 is an exploded isometric view of another example embodiment of the dressing assembly 905, similar to that of FIG. 11, illustrating additional details that may be associated with some embodiments. The dressing assembly 905 of FIG. 12 comprises a support layer, such as the skeleton support structure 910, configured to be disposed on the outer surface 915 of the flange 418 around the manifold 406. The dressing assembly 905 of FIG. 12 may further comprise an upper film layer 1205, which may be disposed on the support layer (e.g. the skeleton support structure 910). In some embodiments, the skeleton support structure 910 may be disposed between the flange 418 and the upper film layer 1205. In some embodiments, the skeleton support structure 910 may comprise a support aperture 1207 configured to allow the skeleton support structure 910 to fit around the manifold 406. In some embodiments, the manifold 406 may project through the support aperture 1207 of the skeleton support structure 910. In some embodiments, the upper film layer 1205 may span the skeleton support structure 910. In some embodiments, the upper film layer 1205 may be similar to the cover 140 or the outer drape layer 715. In some embodiments, the upper film layer 1205 may include a manifold aperture 1210, configured so that when in place, the upper film layer 1205 may be disposed around the manifold 406 (e.g. in a plane above the skeleton support structure 910) but not over the manifold 406. For example, the manifold 406 of the dressing 110 may project through the manifold aperture 1210 of the upper film layer 1205. In some embodiments, the upper film layer 1205 may comprise an adhesive bottom surface 1215, which may be configured to couple both the upper film layer 1205 and the skeleton support structure 910 to the outer surface 915 of the flange 418. For example, the adhesive bottom surface 1215 may permanently attach the skeleton support structure 910 to the flange 418 by adhesion of the upper film layer 1205 to the flange 418 between and around supports of the skeleton support structure 910. In some embodiments, a skeleton support structure 910 similar to that described with respect to FIGS. 11-12 may serve as the support layer for dressings or drapes having internal and/or imbedded support.

In use, method embodiments using a dressing, having a manifold, a cover disposed over the manifold, and a skeleton support structure removably coupled to the cover, may comprise: holding the dressing by the skeleton support structure; applying the dressing to a tissue site by using the skeleton support structure to handle the dressing, wherein the skeleton support structure supports the dressing to prevent the cover from folding over during handling; shaping and adhering the dressing to the tissue site to form a seal for negative-pressure therapy; and/or removing the skeleton support structure from the dressing, while leaving the dressing adhered to the tissue site. In some embodiments, the skeleton support structure may be removed after the dressing has been adhered to the tissue site; and removing the skeleton support structure may not substantially weaken the seal of the dressing to the tissue site. In some embodiments, the skeleton support structure may comprise a projection portion above the manifold, and holding the dressing may comprise grasping the projection portion. In some embodiments, grasping the projection portion may comprise inserting one or more fingers of a user under the projection portion. Some method embodiments may further comprise fluidly coupling the dressing to a negative-pressure source. Some method embodiments may further comprise applying negative pressure to the dressing using the negative-pressure source.

Also disclosed are examples of methods of manufacturing a dressing assembly, similar to the embodiments described herein, which may comprise: providing a dressing having a cover disposed over a manifold; forming a skeleton support structure having a base portion which is configured to support a perimeter of the cover, and a projection portion which is configured to extend from the base portion over the manifold; and removably attaching the base portion of the skeleton support structure to an outer surface of the cover. In some embodiments, forming a skeleton support structure may further comprise: providing a longitudinal support and two lateral supports; and coupling each of the lateral supports to opposite ends of the longitudinal support. In some embodiments, the two lateral supports may extend from the longitudinal support to the cover. In some embodiments, the cover may comprise a flange extending laterally beyond the manifold, and the two lateral supports may extend from the longitudinal support to the flange. In some embodiments, forming a skeleton support structure having a base portion may comprise providing one or more perimeter supports; configuring the one or more perimeter supports to provide support around the perimeter of the flange; coupling the perimeter supports together into the base portion; and coupling the one or more perimeter supports to the longitudinal support with the lateral supports. In some embodiments, the projection portion may comprise the longitudinal support.

The systems, apparatuses, and methods described herein may provide significant advantages. For example, the supported drape or cover for the dressing may improve the ease and accuracy of application of the dressing to the tissue site, and/or may reduce the number of scrapped dressings or drapes. For example, the supported cover may prevent the cover from folding over and adhering to itself (e.g. maintaining the dressing in a flexed state until the dressing is adhered to the tissue site), and this may improve the process for applying the dressing while allowing the dressing to be shaped to the tissue site (e.g. even to curved anatomy). This may also prevent waste, since covers that fold and adhere to themselves may lead to the entire dressing being discarded. Such a reduction in waste may also reduce cost of care. Supported dressing/cover embodiments may also allow for the effective use of larger dressings/covers. In some embodiments, the supported cover may also improve reliability and/or manufacturability by eliminating the need for a kiss-cut process for forming a separate, removable material (since such a cutting process could inadvertently pierce the drape so that it is no longer effectively occlusive due to micro-tears or pin-holes). In some embodiments, the support may also serve as a handle, allowing a user to more effectively handle the dressing. In some instances, the support may allow the user to handle and place the dressing on the tissue site using a single hand, freeing up the user's second hand to apply (e.g. adhere) the dressing to the tissue site (e.g. especially over curved anatomy). Removal of the support after the dressing has been attached over the tissue site may also minimize the profile of the dressing for patient convenience.

If something is described as “exemplary” or an “example”, it should be understood that refers to a non-exclusive example. The terms “about” or “approximately” or the like, when used with a number, may mean that specific number, or alternatively, a range in proximity to the specific number as understood by persons of skill in the art field (for example, +/−10%). Use of broader terms such as “comprises”, “includes”, and “having” should be understood to provide support for narrower terms such as “consisting of”, “consisting essentially of”, and “comprised substantially of”. Use of the term “optionally”, “may”, “might”, “possibly”, “could”, “can”, “would”, “should”, “preferably”, “typically”, “often” and the like with respect to any element, component, feature, characteristic, etc. of an embodiment means that the element, component, feature, characteristic, etc. is not required, or alternatively, the element, component, feature, characteristic, etc. is required, both alternatives being within the scope of the embodiment(s). Such element, component, feature, characteristic, etc. may be optionally included in some embodiments, or it may be excluded (e.g. forming alternative embodiments, all of which are included within the scope of disclosure). Section headings used herein are provided for consistency and convenience, and shall not limit or characterize any invention(s) set out in any claims that may issue from this disclosure. If a reference numeral is used to reference a specific example of a more general term, then that reference numeral may also be used to refer to the general term (or vice versa).

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing, the container, or both may be eliminated or separated from other components for manufacture or sale. In other example configurations, the controller may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims. Also, features, elements, and aspects described with respect to a particular embodiment may be combined with features, elements, and aspects described with respect to one or more other embodiments.

1. A dressing assembly comprising:
a dressing having a manifold and a cover disposed over the manifold, wherein the manifold comprises a tissue-facing surface and an outward-facing surface, the cover is disposed over the outward-facing surface of the manifold, and the cover comprises a flange in proximity to the tissue-facing surface of the manifold; and
a skeleton support structure removably coupled to an outer surface of the flange; wherein the skeleton support structure extends from the outer surface of the flange over the manifold, and is configured to render the flange semi-rigid.

2. The dressing assembly of claim 1, wherein the skeleton support structure comprises a base portion, configured to support the flange, and a projection portion, configured to extend over the manifold of the dressing.

3. The dressing assembly of claim 2, wherein the projection portion of the skeleton support structure is coupled to the base portion and is configured to form a handle for handling the dressing during placement.

4. A dressing assembly comprising:
a dressing having a manifold and a cover disposed over the manifold; and
a support layer coupled to an outer surface of the cover of the dressing.

5. The dressing assembly of claim 4, wherein the support layer comprises a skeleton support structure.

6. The dressing assembly of claim 5, wherein:
the manifold comprises a first surface and a second surface;
the cover is disposed over the second surface of the manifold;
the cover comprises a flange, extending laterally away from the manifold, in proximity to the first surface of the manifold; and
the skeleton support structure is configured to couple to and to support the flange of the cover.

7. (canceled)

8. The dressing assembly of claim 6, wherein the skeleton support structure is non-planar and extends from the outer surface of the flange outward beyond the manifold.

9. The dressing assembly of claim 6, wherein the skeleton support structure comprises a base portion, configured to support the flange, and a projection portion, configured to extend from the outer surface of the flange over the manifold of the dressing.

10. The dressing assembly of claim 6, wherein the skeleton support structure is removably coupled to the outer surface of the flange by a low-tack adhesive.

11. The dressing assembly of claim 6, wherein the skeleton support structure comprises a plurality of supports coupled together into a unitary skeleton support structure.

12. The dressing assembly of claim 6, wherein:
the skeleton support structure comprises a longitudinal support and two lateral supports each extending from the longitudinal support to substantially a perimeter of the flange;
each of the two lateral supports extends from the longitudinal support in proximity to one of two ends of the longitudinal support; and
the two lateral supports each substantially span a width of the cover.

13. The dressing assembly of claim 9, wherein the skeleton support structure further comprises a longitudinal support and two lateral supports, each extending from the longitudinal support to substantially a perimeter of the flange; the base portion comprises one or more perimeter supports configured to span the perimeter of the flange; the lateral supports couple the base portion to the longitudinal support; and the projection portion comprises the longitudinal support.

14. The dressing assembly of claim 9, wherein the skeleton support structure further comprises a longitudinal support and two lateral supports, each extending from the longitudinal support to substantially a perimeter of the flange; the base portion of the skeleton support structure comprises portions of the lateral supports and/or the longitudinal support contacting the flange; and the projection portion of the skeleton support structure comprises portions of the lateral supports and/or longitudinal support disposed over the manifold.

15. (canceled)

16. The dressing assembly of claim 11, wherein each of the supports of the skeleton support structure has a Young's Modulus of about 1-10 GPa.

17. The dressing assembly of claim 5, wherein the skeleton support structure comprises PVC or PU coated copper or steel filament wire with a diameter of about 1-1.5 mm.

18. The dressing assembly of claim 5, wherein the skeleton support structure comprises one or more thin molded components formed of polypropylene, ABS, or PETG and having a thickness of about 0.020-0.200 inch.

19. A method for using a dressing, having a manifold, a cover disposed over the manifold, and a skeleton support structure removably coupled to the cover, comprising:
holding the dressing by the skeleton support structure;
applying the dressing to a tissue site by using the skeleton support structure to handle the dressing, wherein the skeleton support structure supports the dressing to prevent the cover from folding over during handling;
shaping and adhering the dressing to the tissue site to form a seal for negative-pressure therapy; and
removing the skeleton support structure from the dressing, while leaving the dressing adhered to the tissue site.

20. The method of claim 19, wherein:
the skeleton support structure is removed after the dressing has been adhered to the tissue site; and
removing the skeleton support structure does not substantially weaken the seal of the dressing to the tissue site.

21. The dressing assembly of claim 4, wherein the support layer is removably coupled to the outer surface of the cover.

22.-27. (canceled)

28. The dressing assembly of 10, wherein the dressing further comprises an adhesive attachment device configured to couple the dressing to a tissue site for negative-pressure treatment; and the low-tack adhesive coupling the skeleton support structure to the outer surface of the flange has a lower adhesion strength than the adhesive attachment device for the dressing.

29. The dressing assembly of claim 10, wherein the low-tack adhesive has an adhesion strength of about 0.2N-16.1N.

30. (canceled)

31. (canceled)

32. (canceled)

33. The dressing assembly of claim 12, wherein the two lateral supports each extend from opposite sides of the longitudinal support to substantially span the width of the cover.

34. The dressing assembly of claim 12, wherein the skeleton support structure further comprises one or more intermediate lateral supports, spaced between the two ends of the longitudinal support.

35.-37. (canceled)

38. The dressing assembly of claim 12, wherein the dressing comprises one or more wing portions extending laterally away from a longitudinal centerline of the dressing, and for each wing portion, a lateral support spans the wing portion to couple the longitudinal support to the flange at a perimeter of the wing portion.

39.-42. (canceled)

43. The dressing assembly of claim 5, wherein the skeleton support structure is peaked, with two triangle vertices extending from the flange and one triangle vertex located above the manifold.

44. (canceled)

45. The dressing assembly of claim 6, wherein the skeleton support structure is substantially planar, is coupled to the outer surface of the flange, and is configured to render the flange self-supporting.

46. The dressing assembly of claim 45, wherein the skeleton support structure extends around the manifold, but does not extend over the manifold.

47.-52. (canceled)

53. A skeleton support structure for use with a dressing having a manifold with a cover disposed over the manifold and including a flange extending laterally beyond the manifold, the skeleton support structure comprising:
a plurality of supports coupled together and configured to removably couple to an outer surface of the flange of the dressing;
wherein the skeleton support structure is non-planar, is configured to extend from the outer surface of the flange over the manifold, and is configured to render the flange self-supporting.

54.-61. (canceled)

62. A method of manufacturing a dressing assembly, comprising:
providing a dressing having a cover disposed over a manifold;
forming a skeleton support structure having a base portion which is configured to support a perimeter of the cover, and a projection portion which is configured to extend from the base portion over the manifold; and
removably attaching the base portion of the skeleton support structure to an upper surface of the cover.

63.-72. (canceled)

73. The dressing assembly of claim 2, wherein the projection portion is coupled to opposing sides of the base portion and/or extends across opposing sides of the manifold.

74. (canceled)
